(19) United States
(12) Patent Application Publication
(10) Pub. No.: US 2011/0136682 A1
(43) Pub. Date: Jun. 9, 2011

(54) ANTENNARY FUCOSYLATION IN GLYCOPROTEINS FROM CHO CELLS

(75) Inventors: Carlos J. Bosques, Arlington, MA (US); Cuihua Liu, Belmont, MA (US); Jennifer Murphy, Marshfield, MA (US); Nathaniel J. Washburn, Littleton, MA (US); Xiao-Jin Xu, Jamaica Plain, MA (US)

(73) Assignee: MOMENTA PHARMACEUTICALS, INC., Cambridge, MA (US)

(21) Appl. No.: 12/959,811

(22) Filed: Dec. 3, 2010

Related U.S. Application Data
(60) Provisional application No. 61/266,686, filed on Dec. 4, 2009.

(51) Int. Cl. C40B 30/00; C12Q 1/02; C12P 21/00; C07K 14/00; C12N 5/071; C12Q 1/34; C12Q 1/68; C12Q 1/48; G01N 27/447; H01J 49/00; G01J 1/58; G01J 1/42; G01N 21/00; G01R 33/44

(52) U.S. Cl. 506/7; 435/29; 435/69.1; 530/395; 435/358; 435/18; 435/6.1; 435/15; 204/450; 250/281; 250/458.1; 250/372; 356/432; 324/309

(57) ABSTRACT

The present invention provides methods of evaluating CHO cells and producing recombinant glycoproteins.

The present application claims priority to U.S. Provisional patent application Ser. No. 61/266,686, filed on Dec. 4, 2009, the entire disclosure of which is incorporated herein by reference.

### BACKGROUND OF THE INVENTION

Glycoproteins constitute a large portion of the biologics therapeutic market. The glycan structures attached to these proteins are thought to be critical for maintaining their structure, stability and function. Changes in glycosylation can not only affect important properties of a therapeutic glycoprotein product, but can also potentially impact the immunogenic profile of the product. For example, fucosylation has been well documented to have important effects on glycoprotein function. Most commonly fucose is linked via an α-linkage to the C-6 of core GlcNAc. Additionally, in certain cases, fucose moieties can also be added to the C-3 or C-4 of an antennary GlcNAc or Galactose resulting in antennary fucosylated glycan structures. In particular, such antennary fucosylation has been reported to impact the biodistribution (and therefore the in-vivo activity) of therapeutic glycoproteins by increasing targeting to sites of inflammation (Mulligan et al., J. Immunol., 1999, 162: 4952-4959). There are several fucosyl transferase enzymes presumably involved in the formation of these additional linkages (Ma et al., Glycobiology, 2006, 16(12):158R-184R).

Many recombinant therapeutic biopharmaceutical products are produced in Chinese Hamster Ovary (CHO) cells. CHO cells are not known to produce antennary fucosylated structures without introduction of an exogenous transferase (Zhang et al., J. Biol. Chem., 1999, 274(15):10439-10450; Grabenhorst et al., Glycoconjugate J., 1999, 16:81-97).

### SUMMARY OF THE INVENTION

The present invention is based, in part, on the unexpected discovery that glycoproteins produced from CHO cells (e.g., CHO-K, e.g., CHO-K1; CHO DUKX; PA-DUKX; CHO-S; CHO pro3-; CHO pro5; CHO DG44; CHO P12; CHO-DUK-BII or derivatives thereof, that have not been genetically engineered or mutagenized to express an α3/α4 antennary fucosyltransferase, e.g., have not been genetically engineered or mutagenized to express a FucT I, II, III, IV, V, VI, VII, or IX) can contain antennary fucosylated glycan structures, which can affect the biological activity of such glycoproteins that are used for therapeutic purposes; and on the development of methods to screen, identify and quantify such structures in CHO cells.

Antennary or bifucosylated glycan structures in recombinant glycoprotein products administered for therapeutic purposes may affect the biological properties, e.g., the biodistribution, of such products. Considering the potential structural and functional significance of fucosylated structures, it is important to be able to identify and quantify such glycan moieties, not only on a pharmaceutical drug substance or drug product (e.g., in a release test or quality test for a pharmaceutical product), but also during design and development of a product (e.g., in clonal screening and selection, and/or in manufacturing process development), and during commercial manufacturing of a product (e.g., in monitoring manufacturing process quality, product quality and/or batch-to-batch variability). Moreover, the ability to correlate fucosylated structures with the genetic potential of a particular cell line or clonal derivative to enzymatically synthesize such a structure represents an important tool in biologics design and development.

Thus, in a first aspect, the present invention comprises methods for evaluating a Chinese Hamster Ovary (CHO) cell population. In certain embodiments, the testing method includes: (a) providing one or more CHO cells from the population; and (b) evaluating antennary fucosylated glycans produced by said cells. The cells have not been genetically engineered or mutagenized to express an antennary fucosyl tranferase, e.g., have not been genetically engineered to express an α3/α4 antennary fucosyltransferase, e.g., have not been genetically engineered to express FucT I, II, III, IV, V, VI, VII, or IX.

In embodiments, the measuring step may include any of the following: (a) isolating a glycoprotein sample produced by the cells and measuring antennary fucosylated glycans on the isolated glycoprotein sample, (b) isolating a specific glycoprotein composition produced by the cells and measuring the glycans containing antennary fucosylated glycans on the isolated glycoprotein composition, (c) isolating glycans from a glycoprotein sample produced by the cells and measuring the glycans containing antennary fucosylation in the isolated glycans, (d) cleaving monosaccharides from glycans on a glycoprotein sample or on the cell surface of one or more CHO cells, and detecting the fucose monosaccharide released from the antennary fucosylated glycan, (e) providing at least one peptide from a glycoprotein produced by the cells, and measuring the glycans containing antennary fucosylation on the at least one peptide, (f) measuring a relative level of glycans containing antennary fucosylation on the glycoprotein by measuring glycans on the cell surface of the one or more CHO cells, (g) measuring expression of one or more FucT I, II, III, IV, V, VI, VII, or IX gene in the cells.

In some embodiments, the measuring step includes treating a source of glycans, glycoproteins or glycopeptides from the CHO cells with one or more exoglycosidase, e.g., a fucosidase, sialidase, galactosidase, and or hexosaminidase enzyme, followed by analysis of the glycan population thus produced.

In some embodiments, provided methods include preparing a glycoprotein preparation from a culture of the CHO cells, cleaving one or more glycans from the glycoprotein preparation (e.g., with one or more endoglycosidases such as PNGASE-F, or by chemical treatment to remove the glycan) and measuring antennary fucosylation.

Techniques used to measure antennary fucosylated glycans can include one or more of the following methods, and combinations of any of these methods: chromatographic methods, mass spectrometry (MS) methods, electrophoretic methods (such as capillary electrophoresis), nuclear magnetic resonance (NMR) methods, monosaccharide analysis, fluorescence methods, UV-VIS absorbance, enzymatic methods, and use of a detection molecule (such as an antibody or lectin).

In certain embodiments, methods used to detect bifucosylated glycans or antennary fucosylation on a glycan includes high performance anion-exchange chromatography with pulsed amperometric detection (HPAE-PAD). In some embodiments, HPAE-PAD methods can detect antennary fucosylated glycans that are as low in abundance as 0.1% or 0.05% of total glycans.

In certain embodiments, the method used to detect bifucosylated glycans or antennary fucosylation on a glycan is MS/MS.

In some embodiments, methods used provide a qualitative measure. In some embodiments, methods used provide a quantitative measure of glycans containing antennary fucosylated or bifucosylated glycans.

In certain embodiments, methods are conducted during a manufacturing process for a therapeutic glycoprotein by obtaining a sample from a bioreactor containing the CHO cell culture, e.g., to monitor glycan structure during the manufacturing process. In certain embodiments, the measuring step is repeated at least once over time, e.g., the measuring step is repeated at least once, twice, three times or more, during a time period of culture of CHO cells. In some embodiments, the method is conducted on a glycoprotein product produced from CHO cells, e.g., as part of a quality test or release test of the glycoprotein product.

In some embodiments, the measuring step includes comparing the level of antennary fucosylation in a first glycoprotein preparation produced from a first population of CHO cells to the level of glycans containing antennary fucosylation in a second glycoprotein preparation produced from a second population of CHO cells. In some such embodiments, glycans of a glycoprotein preparation from populations of CHO cells cultured under different culture conditions and/or at different times can be determined and compared.

In some embodiments, provided methods may comprise a step of comparing the level of antennary fucosylation to a reference level (e.g., to a control level, or to a range or value in a predetermined product specification). The reference level of antennary fucosylation can be defined in a number of ways, and will vary depending on the selection criteria. To give but a few examples, the target level of antennary fucosylation may be defined as being (a) below a predetermined amount, (b) not more than (NMT) a predetermined amount, (c) at least a predetermined amount, or (d) between predetermined amounts, e.g., within a range of defined acceptable values. In certain embodiments, the reference level will be a level that is below the limit of detection of the method used for the measuring step. In some embodiments, the reference level of antennary fucosylation will be equivalent to the level of antennary fucosylation found in a reference product, e.g., a commercially available reference glycoprotein product, e.g., a commercially available glycoprotein product such as those described herein. In some embodiments, the reference level will be not more than 20% different from the level of antennary fucosylation found in a reference product (e.g., a commercially available glycoprotein product such as those described herein. In some embodiments, the reference level may be that no more than 40% antennary fucosylation is present in a glycoprotein composition, e.g., no more than 30%, 20%, 15, 10%, 5%, 2%, 1%, 0.5% or less. In one embodiment, the level of antennary fucosylation produced by the cells can be measured as the level of glycans containing antennary fucose relative to total amount of glycans in a sample, such as a sample glycoprotein preparation produced from the cells. A skilled artisan could readily convert levels expressed in this way to levels expressed in an alternative way, such as the amount of glycans containing antennary fucose relative to the amount of protein, or as the amount of fucose relative to the amount of other monosaccharides or glycans or protein.

In some embodiments, provided methods include recording the level of antennary glycans or bifucosylated glycans produced by the cells in a print or computer-readable medium, e.g., in a test report, Material Safety Data Sheet (MSDS) or Certificate of Testing or Certificate of Analysis (CofA).

In certain embodiments of provided methods, the measuring step includes use of a detection molecule which is able to detect the presence or absence of antennary fucosylation. In certain embodiments, the detection molecule comprises an antibody that is able to bind to antennary fucose. In some embodiments of the invention, the detection molecule comprises a lectin. In some embodiments, the detection molecule may comprise a fluorescent moiety, or a radioisotope moiety.

A CHO cell population utilized in accordance with the present invention may be a clonal cell population. The CHO cell population may be in culture, e.g., it may be a sample from a bioreactor used to produce a therapeutic glycoprotein. In certain embodiments, the CHO cell population will have been transformed with at least one vector encoding a therapeutic glycoprotein. Therapeutic glycoproteins may be of human, non-human or synthetic origins. Therapeutic glycoproteins may be for treatment of humans or veterinary indications.

In some embodiments, provided methods include a step of evaluating a biological activity of the glycoprotein produced by the cell, e.g., evaluating the receptor affinity, biodistribution or immunogenicity potential of the glycoprotein, e.g., in vitro or in vivo, e.g., in an animal model.

In one embodiment, the CHO cell population is a CHO-K, e.g., CHO-K1; CHO DUKX; PA-DUKX; CHO-S; CHO pro3-; CHO pro5; CHO DG44; CHO P12; CHO-DUK-BII population, or derivative thereof.

In a second aspect, the invention comprises methods for screening one or more Chinese Hamster Ovary (CHO) cells for the ability to produce antennary fucosylated glycans, the method comprising:

(a) providing a plurality of CHO cell populations wherein none of the plurality have been genetically engineered or mutagenized to express an antennary fucosyl tranferase, e.g., have not been genetically engineered to express an α3/α4 antennary fucosyltransferase, e.g., have not been genetically engineered to express FucT I, II, III, IV, V, VI, VII, or IX;

(b) culturing each of the plurality of CHO cell populations under conditions suitable for expression of a glycoprotein expression product;

(c) measuring glycans containing antennary fucosylation produced by each of the plurality of CHO cells, and

(d) selecting one or more of the plurality of CHO cell preparations based on the presence of a target level of antennary fucosylation produced by the selected CHO cell preparation.

The target level of antennary fucosylation can be defined in a number of ways, and will vary depending on the selection criteria. To give but a few examples, the target level of antennary fucosylation may be defined as being (a) below a predetermined amount, (b) not more than (NMT) a predetermined amount, (c) at least a predetermined amount, or (d) between predetermined amounts, e.g., within a range of defined acceptable values. In certain embodiments, the target level will be a level that is below the limit of detection of the method used for the measuring step. In some embodiments, the target level of antennary fucosylation will be equivalent to the level of antennary fucosylation found in a reference product, e.g., a commercially available reference glycoprotein product, e.g., a commercially available glycoprotein product such as those described herein. In some embodiments, the reference level will be not more than 20% different from the level of antennary fucosylation found in a reference product (e.g., a commercially available glycoprotein product such as those described herein. In some embodiments, the reference level may be that no more than 40% antennary fucosylation is present in a glycoprotein composition, e.g., no more than 30%, 20%, 15, 10%, 5%, 2%, 1%, 0.5% or less. In one embodiment, the level of antennary fucosylation produced by the cells can be measured as the level of glycans containing antennary fucose relative to total amount of glycans in a sample, such as a sample glycoprotein preparation produced from the cells. A skilled artisan could readily convert levels expressed in this way to levels expressed in an alternative way, such as the amount of glycans containing antennary fucose relative to the amount of protein, or as the amount of fucose relative to the amount of other monosaccharides or glycans or protein.

The measuring step of the screening method may include any technique for identifying and/or quantifying bifucosylated glycans on a glycoprotein or the level of antennary fucose on a glycan. For example, glycans containing antennary fucosylation may be obtained and measured, e.g., from glycoproteins produced by the CHO cell preparations, from an isolated glycoprotein expression product or composition from the CHO cell preparations, from peptides obtained from a glycoprotein expression product of the CHO cell preparations, from cell surface glycans of the CHO cell preparations, or from glycan preparations obtained from the CHO cell preparations or from a glycoprotein expression product thereof. In certain embodiments, the screening method further comprises the step of isolating a glycoprotein expression product from the cell culture and measuring antennary fucosylation on a glycoprotein produced by the cells in step (c). In certain embodiments, the cell screening method further comprises the step of quantifying the amount of antennary fucosylation present on the glycoprotein expression product. In certain embodiments, step (b) of the cell screening method takes place in a bioreactor, e.g., a commercial bioreactor.

In embodiments, the measuring step may include any of the following: (a) isolating a glycoprotein sample produced by the cells and measuring antennary fucosylated glycans on the isolated glycoprotein sample, (b) isolating a specific glycoprotein composition produced by the cells and measuring the glycans containing antennary fucosylated glycans on the isolated glycoprotein composition, (c) isolating glycans from a glycoprotein sample produced by the cells and measuring the glycans containing antennary fucosylation in the isolated glycans, (d) cleaving monosaccharides from glycans on a glycoprotein sample or on the cell surface of one or more CHO cells, and detecting the fucose monosaccharide released from the antennary fucosylated glycan, (e) providing at least one peptide from a glycoprotein produced by the cells, and measuring the glycans containing antennary fucosylation on the at least one peptide, (f) measuring a relative level of glycans containing antennary fucosylation on the glycoprotein by measuring glycans on the cell surface of the one or more CHO cells, (g) measuring expression of one or more FucT I, II, III, IV, V, VI, VII, or IX gene in the cells.

In some embodiments, methods used provide a quantitative measure of glycans containing antennary fucosylated or bifucosylated glycans. In some embodiments, the method used provides a qualitative measure.

Each of the plurality of CHO cell populations may comprise a different CHO strain population, a different clonal cell population, or different samples (e.g., samples taken over time) from a cell culture during a manufacturing process for a therapeutic glycoprotein. In certain embodiments, each of the plurality of CHO cell populations will have been transformed with at least one vector encoding a therapeutic glycoprotein, e.g., a human therapeutic glycoprotein. In certain embodiments of the cell screening method, the glycoprotein expression product is a secreted glycoprotein expressed from CHO cells.

In some embodiments, glycans containing antennary fucosylation can be measured as the level of glycans containing antennary fucose relative to total amount of glycans in a sample, such as a sample glycoprotein preparation produced from the cells. A skilled artisan could readily convert levels expressed in this way to levels expressed in an alternative way, such as the amount of glycans containing antennary fucose relative to the amount of protein, or as the amount of fucose relative to the amount of other monosaccharides or glycans or protein.

In some embodiments, provided methods include recording the level of antennary fucosylation produced by one or more of the plurality of the cells in a print or computer-readable medium, e.g., in a test report.

In one embodiment, the CHO cell population is a CHO-K, e.g., CHO-K1; CHO DUKX; PA-DUKX; CHO-S; CHO pro3-; CHO pro5; CHO DG44; CHO P12; CHO-DUK-BII population, or derivative thereof.

In a third aspect, the invention includes a method for evaluating a glycoprotein composition. The method includes measuring the amount of antennary fucosylation present in a glycoprotein composition, wherein the glycoprotein composition was produced in CHO host cells. The CHO host cells were not genetically engineered or mutagenized to express an antennary fucosyl tranferase, e.g., were not genetically engineered to express an α3/α4 antennary fucosyltransferase, e.g., were not genetically engineered to express FucT I, II, III, IV, V, VI, VII, or IX.

In some embodiments, provided methods include recording the level of antennary fucosylation present in the glycoprotein composition in a print or electronic record, e.g., a test report or Material Safety Data Sheet (MSDS) or Certificate of Testing or Certificate of Analysis (CofA).

In some embodiments, provided methods include comparing the measured level of antennary fucosylation present in the glycoprotein composition with a reference level, such as a control level or to a range or value in a predetermined product specification or reference specification. The reference level can be a specification (e.g., an FDA label or Physician's Insert) or quality criterion for a pharmaceutical preparation containing the glycoprotein composition. The reference level of antennary fucosylation can be defined in a number of ways, and will vary depending on the selection criteria. To give but a few examples, the target level of antennary fucosylation may be defined as being (a) below a predetermined amount, (b) not more than (NMT) a predetermined amount, (c) at least a predetermined amount, or (d) between predetermined amounts, e.g., within a range of defined acceptable values. In certain embodiments, the reference level will be a level that is below the limit of detection of the method used for the measuring step. In some embodiments, the reference level of antennary fucosylation will be equivalent to the level of antennary fucosylation found in a reference product, e.g., a commercially available reference glycoprotein product, e.g., a commercially available glycoprotein product such as those described herein. In some embodiments, the reference level will be not more than 20% different from the level of antennary fucosylation found in a reference product (e.g., a commercially available glycoprotein product such as those described herein. In some embodiments, the reference level may be that no more than 40% antennary fucosylation is present in a glycoprotein composition, e.g., no more than 30%, 20%, 15, 10%, 5%, 2%, 1%, 0.5% or less. In one embodiment, the level of antennary fucosylation produced by the cells can be measured as the level of glycans containing antennary fucose relative to total amount of glycans in a sample, such as a sample glycoprotein preparation produced from the cells. A skilled artisan could readily convert levels expressed in this way to levels expressed in an alternative way, such as the amount of glycans containing antennary fucose relative to the amount of protein, or as the amount of fucose relative to the amount of other monosaccharides or glycans or protein.

In some embodiments, the reference level or quality criterion is that no more than 40% antennary fucosylation present in a glycoprotein composition be present, e.g., no more than 30%, 20%, 15, 10%, 5%, 2%, 1%, 0.5% or less. In one embodiment, the level of antennary fucosylation produced by the cells can be measured as the level of glycans containing antennary fucose relative to total amount of glycans in a sample, such as a sample glycoprotein preparation produced from the cells. A skilled artisan could readily convert levels expressed in this way to levels expressed in an alternative way, such as the amount of glycans containing antennary fucose relative to the amount of protein, or as the amount of fucose relative to the amount of other monosaccharides or glycans or protein.

In one embodiment, one or more of the plurality of CHO cell population is selected from: a CHO-K, e.g., CHO-K1; CHO DUKX; PA-DUKX; CHO-S; CHO pro3-; CHO DG44; CHO pro5; CHO P12; CHO-DUK-BII population, or derivative thereof.

In a fourth aspect, the invention features a method of making a therapeutic glycoprotein. The method includes (a) providing a CHO cell (e.g., a CHO-K cell, e.g., CHO-K1 cell, or other derivative thereof, or another CHO cell strain described herein) that has been genetically engineered to express an exogenous therapeutic glycoprotein, (b) culturing the cell to produce a therapeutic glycoprotein, e.g., in a bioreactor, (c) purifying the therapeutic glycoprotein from the cell culture, e.g., to produce a therapeutic glycoprotein API, and (d) evaluating, measuring, or monitoring the level of antennary fucosylation present in the therapeutic glycoprotein. The CHO cell has not been genetically engineered or mutagenized to express an antennary fucosyl tranferase, e.g., has not been genetically engineered or mutagenized to express an α3/α4 antennary fucosyltransferase, e.g., FucT I, II, III, IV, V, VI, VII, or IX.

In one embodiment, the level of antennary fucosylation can be evaluated, measured or monitored during one or more of: the cell culture step, the purification step, and in the purified glycoprotein product.

In embodiments, the evaluation, measuring or monitoring step may include any of the following: (a) isolating a glycoprotein sample produced by the cells and measuring antennary fucosylated glycans on the isolated glycoprotein sample, (b) isolating a specific glycoprotein composition produced by the cells and measuring the glycans containing antennary fucosylated glycans on the isolated glycoprotein composition, (c) isolating glycans from a glycoprotein sample produced by the cells and measuring the glycans containing antennary fucosylation in the isolated glycans, (d) cleaving monosaccharides from glycans on a glycoprotein sample or on the cell surface of one or more CHO cells, and detecting antennary fucosylation from the cleaved monosaccharides, (e) providing at least one peptide from a glycoprotein produced by the cells, and measuring the glycans containing antennary fucosylation on the at least one peptide, (f) measuring a relative level of glycans containing antennary fucosylation on the glycoprotein by measuring glycans on the cell surface of the one or more CHO cells, (g) measuring expression of one or more FucT I, II, III, IV, V, VI, VII, or IX gene in the cells.

In some embodiments, the measuring step includes treating a source of glycans, glycoproteins or glycopeptides from the CHO cells with one or more exoglycosidase, e.g., a fucosidase, sialidase, galactosidase, and or hexosaminidase enzyme, followed by analysis of the glycan population thus produced.

In some embodiments, provided methods include preparing a glycoprotein preparation from a culture of the CHO cells, cleaving one or more glycans from the glycoprotein preparation (e.g., with one or more endoglycosidases such as PNGASE-F, or by chemical treatment to remove the glycan) and measuring antennary fucosylation.

The technique used to measure antennary fucosylated glycans can include one or more of the following methods, and combinations of any of these methods: chromatographic methods, mass spectrometry (MS) methods, electrophoretic methods (such as capillary electrophoresis), nuclear magnetic resonance (NMR) methods, monosaccharide analysis, fluorescence methods, UV-VIS absorbance, enzymatic methods, and use of a detection molecule (such as an antibody or lectin).

In certain embodiments, provided methods used to detect bifucosylated glycans or antennary fucosylation on a glycan include high performance anion-exchange chromatography with pulsed amperometric detection (HPAE-PAD). In some embodiments, the HPAE-PAD method can detect antennary fucosylated glycans that are as low in abundance as 0.1% or 0.05% of total glycans.

In certain embodiments, the method used to detect bifucosylated glycans or antennary fucosylation on a glycan is MS/MS.

In some embodiments, methods used provide a qualitative measure. In some embodiments, the methods used provide a quantitative measure of glycans containing antennary fucosylated or bifucosylated glycans.

In certain embodiments, the evaluation, measuring or monitoring step is repeated at least once, twice, three times or more, during the time period of culture of the CHO cells. In some embodiments, the method is conducted on the glycoprotein product produced from the CHO cells, e.g., as part of a quality test or release test of the glycoprotein product.

In some embodiments, provided methods may comprise a step of comparing the level of antennary fucosylation to a reference level (e.g., to a control level, or to a range or value in a predetermined product specification). The reference level of antennary fucosylation can be defined in a number of ways, and will vary depending on the selection criteria. For example, the target level of antennary fucosylation may be defined as being (a) below a predetermined amount, (b) not more than (NMT) a predetermined amount, (c) at least a predetermined amount, or (d) between predetermined amounts, e.g., within a range of defined acceptable values. In certain embodiments, the reference level will be a level that is below the limit of detection of the method used for the measuring step. In some embodiments, the reference level of antennary fucosylation will be equivalent to the level of antennary fucosylation found in a reference product, e.g., a commercially available reference glycoprotein product, e.g., a commercially available glycoprotein product such as those described herein. In some embodiments, the reference level will be not more than 20% different from the level of antennary fucosylation found in a reference product (e.g., a commercially available glycoprotein product such as those described herein. In some embodiments, the reference level may be that no more than 40% antennary fucosylation is present in a glycoprotein composition, e.g., no more than 30%, 20%, 15, 10%, 5%, 2%, 1%, 0.5% or less. In one embodiment, the level of antennary fucosylation produced by the cells can be measured as the level of glycans containing antennary fucose relative to total amount of glycans in a sample, such as a sample glycoprotein preparation produced from the cells. A skilled artisan could readily convert levels expressed in this way to levels expressed in an alternative way, such as the amount of glycans containing antennary fucose relative to the amount of protein, or as the amount of fucose relative to the amount of other monosaccharides or glycans or protein.

In some embodiments, provided methods include recording the level of antennary glycans or bifucosylated glycans produced by the cells in a print or computer-readable medium, e.g., in a test report, Material Safety Data Sheet (MSDS) or Certificate of Testing or Certificate of Analysis (CofA).

In certain embodiments of methods provided herein, the evaluating, measuring or monitoring step includes use of a detection molecule which is able to detect the presence or absence of antennary fucosylation. In certain embodiments, the detection molecule comprises an antibody that is able to bind to antennary fucose. In some embodiments of the invention, the detection molecule comprises a lectin. In some embodiments, the detection molecule comprises a fluorescent moiety, or a radioisotope moiety.

Therapeutic glycoproteins may be of human, non-human or synthetic origins. Therapeutic glycoproteins may be for treatment of humans or veterinary indications.

In some embodiments, provided methods include a step of evaluating a biological activity of the glycoprotein produced by the cell, e.g., evaluating the biodistribution or immunogenicity potential of the glycoprotein, e.g., in vitro or in vivo, e.g., in an animal model.

In one embodiment, the CHO cell population is a CHO-K, e.g., CHO-K1; CHO DUKX; PA-DUKX; CHO-S; CHO pro3-; CHO pro5; CHO DG44; CHO P12; CHO-DUK-BII population, or derivative thereof.

Techniques used to measure antennary fucosylation can include one or more of: a chromatographic method, e.g., High performance Anion Exchange chromatography using Pulsed Amperometric Detection (HPAEC-PAD); mass spectrometry (MS) methods, e.g., MS/MS; electrophoretic methods (such as capillary electrophoresis); nuclear magnetic resonance (NMR) methods; monosaccharide analysis; fluorescence methods; UV-VIS absorbance; enzymatic methods; use of a detection molecule (such as an antibody or lectin).

In a fifth aspect, the invention features a method of producing a glycoprotein having a target level of antennary fucosylation. The method includes (a) defining a target level of antennary fucosylation to be present in a therapeutic glycoprotein, and (b) selecting a CHO cell (e.g., a CHO-K1 or derivative thereof, or other CHO cell strain described herein) as a host cell for production of the therapeutic glycoprotein if the target level of antennary fucosylation is greater than zero, (c) genetically engineering the selected CHO cell to express the therapeutic glycoprotein, and (d) culturing the genetically engineered CHO cell to produce the therapeutic glycoprotein. The CHO cell is not genetically engineered or mutagenized to express an α3/α4 antennary fucosyltransferase (e.g., FucT I, II, III, IV, V, VI, VII, or IX). The method may also include, after step (a), screening CHO-K1 cells clones for a pre-specified level of antennary fucosylation (e.g., by screening for levels of expression of FucT I, II, III, IV, V, VI, VII, or IX. The method may also include, before step (a), measuring the level of antennary fucosyltransferase in a target glycoprotein or reference glycoprotein, to thereby define a target level of antennary fucosylation.

In some embodiments, the target level of antennary fucosylation corresponds to the level present in a commercial version of the therapeutic glycoprotein, e.g., a commercial glycoprotein described herein. In another embodiment, the target level of antennary fucosylation corresponds to a level greater than that present in a commercial version of the therapeutic glycoprotein, e.g., a commercial glycoprotein described herein. In yet another embodiment, the target level of antennary fucosylation corresponds to a level less than that present in a commercial version of the therapeutic glycoprotein, e.g., a commercial glycoprotein described herein.

In some embodiments, provided methods include measuring the level of antennary fucosylation in the produced glycoprotein. The measuring step may include any of the following: (a) isolating a glycoprotein sample produced by the cells and measuring antennary fucosylated glycans on the isolated glycoprotein sample, (b) isolating a specific glycoprotein composition produced by the cells and measuring the glycans containing antennary fucosylated glycans on the isolated glycoprotein composition, (c) isolating glycans from a glycoprotein sample produced by the cells and measuring the glycans containing antennary fucosylation in the isolated glycans, (d) cleaving monosaccharides from glycans on a glycoprotein sample or on the cell surface of one or more CHO cells, and detecting antennary fucosylation from the cleaved monosaccharides, (e) providing at least one peptide from a glycoprotein produced by the cells, and measuring the glycans containing antennary fucosylation on the at least one peptide, (f) measuring a relative level of glycans containing antennary fucosylation on the glycoprotein by measuring glycans on the cell surface of the one or more CHO cells, (g) measuring expression of one or more FucT I, II, III, IV, V, VI, VII, or IX gene in the cells.

In some embodiments, provided methods include cleaving one or more glycans from the produced glycoprotein preparation (e.g., with one or more endoglycosidases such as PNGASE-F, or by chemical treatment to remove the glycan) and measuring antennary fucosylation.

Techniques used to measure antennary fucosylated glycans can include one or more of the following methods, and combinations of any of these methods: chromatographic methods, mass spectrometry (MS) methods, electrophoretic methods (such as capillary electrophoresis), nuclear magnetic resonance (NMR) methods, monosaccharide analysis, fluorescence methods, UV-VIS absorbance, enzymatic methods, and use of a detection molecule (such as an antibody or lectin).

In certain embodiments, methods used to detect bifucosylated glycans or antennary fucosylation on a glycan includes high performance anion-exchange chromatography with pulsed amperometric detection (HPAE-PAD). In some embodiments, HPAE-PAD methods can detect antennary fucosylated glycans that are as low in abundance as 0.1% or 0.05% of total glycans.

In certain embodiments, the method used to detect bifucosylated glycans or antennary fucosylation on a glycan is MS/MS.

In some embodiments, methods used provide a qualitative measure. In some embodiments, methods used provide a quantitative measure of glycans containing antennary fucosylated or bifucosylated glycans.

In certain embodiments, the evaluation, measuring or monitoring step is repeated at least once, twice, three times or more, during the time period of culture of the CHO cells. In some embodiments, the method is conducted on the glycoprotein product produced from the CHO cells, e.g., as part of a quality test or release test of the glycoprotein product.

In some embodiments, provided methods may comprise a step of comparing the level of antennary fucosylation to a reference level (e.g., to a control level, or to a range or value in a predetermined product specification).

In some embodiments, the level of antennary fucosylation produced by the cells can be measured as the level of glycans containing antennary fucose relative to total amount of glycans in a sample, such as a sample glycoprotein preparation produced from the cells. A skilled artisan could readily convert levels expressed in this way to levels expressed in an alternative way, such as the amount of glycans containing antennary fucose relative to the amount of protein, or as the amount of fucose relative to the amount of other monosaccharides or glycans or protein.

In some embodiments, provided methods include recording the level of antennary glycans or bifucosylated glycans produced by the cells in a print or computer-readable medium, e.g., in a test report, Material Safety Data Sheet (MSDS) or Certificate of Testing or Certificate of Analysis (CofA).

Therapeutic glycoproteins may be of human, non-human or synthetic origins. Therapeutic glycoproteins may be for treatment of humans or veterinary indications.

In some embodiments, provided methods include a step of evaluating a biological activity of the glycoprotein produced by the cell, e.g., evaluating the biodistribution or immunogenicity potential of the glycoprotein, e.g., in vitro or in vivo, e.g., in an animal model.

In one embodiment, the CHO cell population is a CHO-K, e.g., CHO-K1; CHO DUKX; PA-DUKX; CHO-S; CHO pro3-; CHO pro5; CHO DG44; CHO P12; CHO-DUK-BII population, or derivative thereof.

Technique used to measure antennary fucosylation can include one or more of: a chromatographic method, e.g., High performance Anion Exchange chromatography using Pulsed Amperometric Detection (HPAEC-PAD); mass spectrometry (MS) methods, e.g., MS/MS; electrophoretic methods (such as capillary electrophoresis); nuclear magnetic resonance (NMR) methods; monosaccharide analysis; fluorescence methods; UV-VIS absorbance; enzymatic methods; use of a detection molecule (such as an antibody or lectin).

In a sixth aspect, the invention includes a recombinant glycoprotein produced in CHO-K cells (e.g., CHO-K1, or other derivative thereof) where the recombinant glycoprotein has a different level of antennary fucosylation than a reference glycoprotein that has the same or highly similar amino acid sequence, and where the cells have not been modulated to express an antennary fucosyl transferase, e.g., have not been genetically engineered or mutagenized to express an antennary fucosyl tranferase, e.g., have not been genetically engineered to express an α3/α4 antennary fucosyltransferase, e.g., have not been genetically engineered to express FucT I, II, III, IV, V, VI, VII, or IX. In one embodiment, the reference glycoprotein is not produced in CHO-K1 cells. A highly similar amino acid sequence, as used herein, is at least 90%, 91%, 92%, 93%, 94%, 95%, 96%, 97%, 98%, or 99% identical.

In some embodiments, the reference glycoprotein is a commercially available therapeutic glycoprotein, e.g., a therapeutic glycoprotein disclosed in Table 2.

The recombinant glycoprotein produced by the methods in accordance with the present invention may have a higher or lower level of antennary fucosylation than the reference glycoprotein, e.g., at least 2%, 5%, 10%, 15%, 20%, 25%, 30%, 35%, 40%, 45%, 50%, 60%, 70%, 80% higher or lower level, e.g., as measured as a percent of total glycans.

In a seventh aspect, the invention features an isolated population of CHO-K1 cells, wherein the cells have not been genetically engineered or mutagenized to express an α3/α4 antennary fucosyltransferase (e.g., FucT I, II, III, IV, V, VI, VII, or IX), and wherein the population has been selected (e.g., through clonal screening), for high level expression of an α3/α4 antennary fucosyltransferase. In one embodiment, the level of expression of FucT I, II, III, IV, V, VI, VII, or IX in the isolated population is higher relative to the level of expression in a parent strain or a control clone. In another embodiment, the level of expression of FucT I, II, III, IV, V, VI, VII, or IX in the isolated population is higher relative to the level of expression of a control gene, e.g., based on a Cp value, e.g., as determined by qPCR.

In some embodiments, the isolated population of CHO-K1 cells has 5%, 10%, 20%, 50%, 100%, 200%, 300%, 400%, or 500% higher levels of expression of an α3/α4 antennary fucosyltransferase than a control (e.g. non-selected) cell population.

In an eighth aspect, the invention features a method of evaluating antennary fucosylation using high performance anion-exchange chromatography with pulsed amperometric detection (HPAE-PAD). In some embodiments, the method detects less than as about 0.5%, 0.4%, 0.2%, 0.1%, 0.05% antennary fucosylation in a glycoprotein or glycan sample, e.g., relative to total glycan.

### BRIEF DESCRIPTION OF THE FIGURES

FIG. 1 is a cartoon showing a bifucosylated glycan with an antennary fucose in addition to core fucose present on N-linked and O-linked glycans. Only FucT VIII is known in the literature to be naturally expressed in CHO cell lines.

FIG. 2 is a set of HPAEC-PAD glycan profiles from a model receptor-Ig fusion protein produced from two different CHO clones. FIG. 2A shows a typical profile and FIG. 2B shows an atypical profile that includes an additional peak relative to a typical profile.

FIG. 3 is a MALDI mass spectrometry profile of the atypical peak of FIG. 2, confirming the presence of a bifucosylated glycan.

FIG. 4 is an MS-MS profile of the atypical peak of FIG. 2, confirming the presence of a bifucosylated glycan.

FIG. 5 is a bar graph showing the percent of glycans containing branched fucose (relative to total glycans) from various clones from 3 different CHO cell lines expressing an exemplary protein (CTLA4-Ig), as determined by HPAEC-PAD.

### DEFINITIONS

Unless otherwise defined hereinbelow, all terms used herein are used in their ordinary meaning, as would be understood by one skilled in the art.

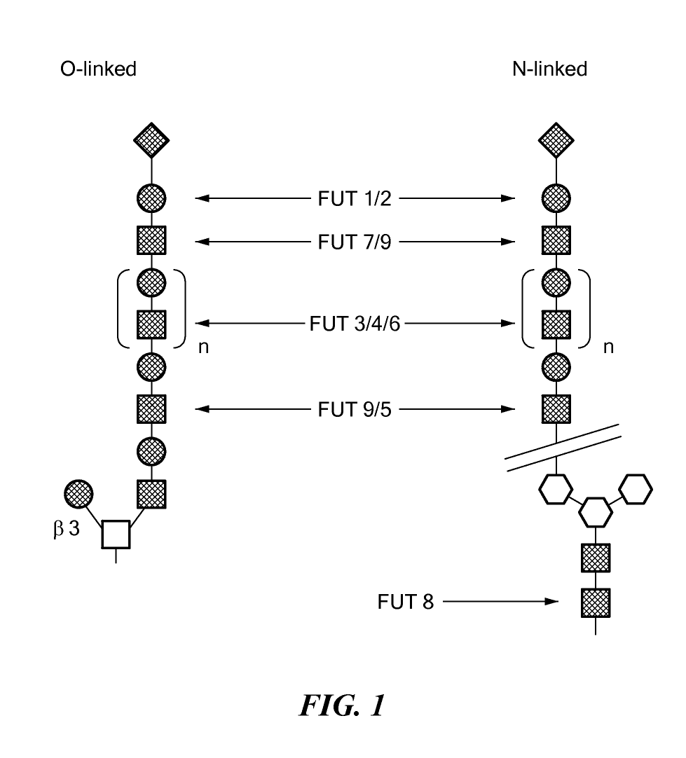

“Antennary fucose-containing glycan”, “branched fucose-containing glycan”: These terms, as used herein, interchangeably describe a glycan that contains a fucose moiety on a branch of an O or N-linked glycan. A branch refers to the portion of the N-glycan that is distal to (away from the reducing end) of the trimannosyl core. A branch on an O-linked glycan refers to a portion of the glycan that is distal (away from the reducing end) or the core GalNAc-Ser/Thre. An example of an N-linked antennary fucose containg glycan is illustrated in FIG. 1. An antennary fucose may be present in addition to the core fucose (as illustrated in FIG. 1) while in others the core fucose may be absent. More than one branch fucose moiety may be present on one glycan structure.

Bifucosylated glycan, As used herein, the term “bifucosylated glycan” refers to a glycan that contains a fucose linked to the core GlcNAc as well as an antennary fucose linked to a branch. An example of a bifucosylated glycan is illustrated in FIG. 1. As used herein, bifucosylated glycan does not refer to two fucose moieties linked to a branch or two fucose linked to the core GlcNAc.

Bioreactor: As used herein, the term “bioreactor” is an apparatus or system used for culturing living cells. A bioreactor can be used to grow living cells (e.g., mammalian cells such as CHO cells) that produce a therapeutic glycoprotein. Typically, a bioreactor includes a vessel for cell growth and, optionally, one or more of: ports for adding or removing medium, ports for adding or removing gas or air, and ports that allow sensors to sample the space inside the vessel. Bioreactors range in size from small laboratory containers of 100 ml or less to large, industrial or commercial-scale tanks having a volume capacity from 1 L to 10,000 L or more.

Detection, Detecting: As used herein, the terms “detecting,” “detection” and “detecting means” are used interchangeably to refer to the determination of whether a particular chemical moiety, such as an antennary fucose residue, is present or absent in or on a compound, composition, cell or cell population. The detecting means may involve a selectable marker, or an identifiable characteristic such as a fluorescent or radioactive moiety, and may involve labeling of a reagent, compound, cell or cell population. Detection can also refer to the analysis of a compound, composition, cell or cell population, using such techniques as mass spectrometry or related methods, electrophoretic methods, nuclear magnetic resonance, chromatographic methods, or combinations of the above, to determine the presence or absence of a chemical moiety in or on a compound, composition, cell or cell population. Detection may also involve quantification of the absolute or relevant levels of the chemical moiety being detected.

Glycan: As is known in the art and used herein “glycans” are sugars. Glycans can be monomers or polymers of sugar residues, but typically contain at least three sugar residues, and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.). The term “glycan” includes homo and heteropolymers of sugar residues. The term “glycan” also encompasses a glycan component of a glycoprotein (e.g., of a glycoprotein, glycolipid, proteoglycan, etc.). The term also encompasses free glycans, e.g., glycans that have been cleaved or otherwise released from a glycoprotein.

Glycan preparation: The term “glycan preparation” as used herein refers to a set of glycans obtained according to a particular production method. In some embodiments, glycan preparation refers to a set of glycans obtained from a glycoprotein preparation (see definition of glycoprotein preparation below). In some embodiments, a glycan preparation includes glycoproteins. In some embodiments, a glycan preparation includes released glycans.

Glycoprotein: As used herein, the term “glycoprotein” refers to a “protein” (as defined herein) that contains a peptide backbone covalently linked to one or more sugar moieties (i.e., glycans). As is understood by those skilled in the art, the peptide backbone typically comprises a linear chain of amino acid residues. The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. In certain embodiments, sugar moieties may include sulfate and/or phosphate groups. Alternatively or additionally, sugar moieties may include acetyl, glycolyl, propyl or other alkyl modifications. In certain embodiments, glycoproteins contain O-linked sugar moieties; in certain embodiments, glycoproteins contain N-linked sugar moieties.

Glycoprotein preparation: A “glycoprotein preparation,” as that term is used herein, refers to a set of individual glycoprotein molecules, each of which comprises a polypeptide having a particular amino acid sequence (which amino acid sequence includes at least one glycosylation site) and at least one glycan covalently attached to the at least one glycosylation site. Individual molecules of a particular glycoprotein within a glycoprotein preparation typically have identical amino acid sequences but may differ in the occupancy of the at least one glycosylation sites and/or in the identity of the glycans linked to the at least one glycosylation sites. That is, a glycoprotein preparation may contain only a single glycoform of a particular glycoprotein, but more typically contains a plurality of glycoforms. Different preparations of the same glycoprotein may differ in the identity of glycoforms present (e.g., a glycoform that is present in one preparation may be absent from another) and/or in the relative amounts of different glycoforms.

Glycosidase: The term “glycosidase” as used herein refers to an agent that cleaves a covalent bond between sequential sugars in a glycan or between the sugar and the backbone moiety (e.g., between sugar and peptide backbone of glycoprotein). In some embodiments, a glycosidase is an enzyme. In certain embodiments, a glycosidase is a protein (e.g., a protein enzyme) comprising one or more polypeptide chains. In certain embodiments, a glycosidase is a chemical cleavage agent, e.g., hydrazine.

N-glycan: The term “N-glycan,” as used herein, refers to a polymer of sugars that has been released from a glycoprotein but was formerly linked to a glycoprotein via a nitrogen linkage (see definition of N-linked glycan below).

N-linked glycans: N-linked glycans are glycans that are linked to a glycoprotein via a nitrogen linkage. A diverse assortment of N-linked glycans exists, but is typically based on the common core pentasaccharide (Man)₃(GlcNAc)(GlcNAc).

Modulate: The term “modulate” as used herein refers to the ability to of an actor to control, within prescribed limits, the value of a parameter, such as the level of antennary fucose residues present in a glycoprotein composition. Thus, in some embodiments, the level of antennary fucose residues may be modulated so that it remains within prescribed limits. In some embodiments, the level of antennary fucose residues may be modulated so that it does not exceed more than 40%, 30%, 25%, 15%, 10%, 5%, 2%, 1%, 0.5%, 0.1% or less of the total N-glycans present in a glycoprotein composition. In other embodiments, the level of antennary fucose residues may be modulated so that it does not vary by more than 25%, 10.0%, 5.0%, 1.0%, 0.5% or 0.1% of a prescribed or desired level.

Protease: The term “protease” as used herein refers to an agent that cleaves a peptide bond between sequential amino acids in a polypeptide chain. In some embodiments, a protease is an enzyme (i.e., a proteolytic enzyme). In certain embodiments, a protease is a protein (e.g., a protein enzyme) comprising one or more polypeptide chains. In certain embodiments, a protease is a chemical cleavage agent.

Providing: The term “providing” as used herein refers to an actor obtaining a subject item, such as a CHO cell, CHO cell preparation, or glycoprotein preparation, from any source including, but not limited to, obtaining by the actor's own manufacture or by the actor's receiving the item from another party. For example, a CHO cell preparation is provided if it is made or received by any machine, person, or entity. In some embodiments, a CHO cell preparation may be received by a machine, which may then perform one or more tests, processes, or refinements of the glycoprotein preparation. In some embodiments, a CHO cell preparation may be received by a person. In some embodiments, a CHO cell preparation may be received from an outside entity. In some embodiments, a CHO cell preparation may be received by a person or business performing characterization services for a second person or business.

### DETAILED DESCRIPTION OF CERTAIN EMBODIMENTS

It has been previously reported that antennary fucosylation is not naturally present in recombinant glycoproteins produced by Chinese Hamster Ovary (CHO) cells (Zhang et al., J. Biol. Chem., 1999, 274(15):10439-10450; Grabenhorst et al., Glycoconjugate J., 1999, 16:81-97). The present disclosure is based, at least in part, on the unexpected finding that antennary fucosylated glycan structures can be found on glycoproteins produced by CHO cells, and thus it is important to identify, monitor and control this aspect of glycan structure when using CHO cells to produce therapeutic products. Thus, the present disclosure provides methods of evaluating antennary fucosylation in CHO cells, and evaluating glycoproteins made in CHO cells for antennary fucosylation. Also provided are related methods of making glycoprotein products in CHO cells (e.g., products having different levels of antennary fucosylation) as well as related glycoprotein preparations, and certain isolated CHO cells.

### Production of Therapeutic Glycoproteins

Methods of making recombinant therapeutic glycoproteins described herein are known in the art. See for example Current Protocols in Molecular Biology (2007, John Wiley and Sons, Inc., Print ISSN: 1934-3639); Current Protocols in Cell Biology (2007, John Wiley and Sons, Inc., Print ISSN: 1934-2500); Current Protocols in Protein Science (2007, John Wiley and Sons, Inc., Print ISSN: 1934-3655); Wurm, Production of recombinant protein therapeutics in cultivated mammalian cells (2004) Nature Biotech. 22:1393-1398; Therapeutic Proteins: Methods and Protocols, Smales and James, eds. (2005, Humana Press, ISBN-10: 1588293904).

Vectors and Host Cells:

Vectors (e.g., expression vectors comprising a coding sequence for a therapeutic glycoprotein) can include a plasmid, cosmid or viral vector. The vector can be capable of autonomous replication or it can integrate into a host DNA. Preferably a recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides. Expression vectors comprising a coding sequence for a therapeutic glycoprotein are preferably able to dive expression of the glycoprotein in a CHO cell. When used in mammalian cells such as CHO, the expression vector's control functions can be provided by viral regulatory elements. For example, commonly used promoters are derived from polyoma, Adenovirus 2, cytomegalovirus and Simian Virus 40. In one embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off” from Clontech Inc., CA.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell (e.g., a host cell containing a vector described herein) is preferably a CHO cell. The CHO cell used in methods of the invention are not genetically engineered to express an α3/α4 antennary fucosyltransferase. For example, in some embodiments, CHO cells used in accordance with the present invention have not been genetically engineered to express a α3/α4 antennary fucosyltransferase selected from the group consisting of FucT I, II, III, IV, V, VI, VII, VIII, IX, and combinations thereof. Alternatively or additionally, in some embodiments, CHO cells used in methods described herein have not been mutagenized to express an α3/α4 antennary fucosyltransferase, for example selected from the group consisting of FucT I, II, III, IV, V, VI, VII, VIII, IX, and combinations thereof. It will be appreciated that not all FucT, I, II, III, IV, V, VI, VII, VIII, and IX polypeptides may have α3/α4 antennary fucosyltransferase activity; for example at least some FucT VIII polypeptides have been reported to be core fucosyltransferases having alpha 1,6-fucosyltransferase activity (see, for example, Yanagidani, et al. J Biochem 121(3):626-32 1997). In certain contemplated embodiments, the FucT I, II, III, IV, V, VI, VII, VIII, and/or IX polypeptides whose engineered or mutagenized expression is excluded in accordance with the present invention are only those that show α3/α4 antennary fucosyltransferase activity.

CHO cells that can be used in the methods include: CHO-K, e.g., CHO-K1 (ATCC CRL-9618); CHO DUKX (ATCC CRL-9096); PA-DUKX; CHO-S; CHO pro3-; CHO pro5 (ATCC CRL-1781); CHO DG44 (Urlaub et al., (1983) Cell 33:405-412); CHO P12; CHO-DUK-BII, or derivatives thereof. Other suitable CHO host cells are known to those skilled in the art.

Protein Production and Purification:

A wide array of flasks, bottles, reactors, and controllers allow the production and scale up of cell culture systems. Cells can be grown, for example, as batch, fed-batch, perfusion, or continuous cultures, typically in bioreactors (e.g., stir-tank bioreactors, airlift bioreactors, roller bottles, immobilized cell bioreactors, spinner cultures, shaker flasks, suspension cell cultures, multistage bioreactors, centrifugal bioreactor, and cell culture bags. Microcarrier beads can be used to increase cell densities.

Production parameters including purification and formulation can be used to produce a glycoprotein preparation with a desired glycan property or properties as described herein. Various purification processes can be used to prejudice the glycan characteristics of the purified glycoprotein preparation. For example, affinity based methods, charged based methods, polarity based methods and methods that distinguish based upon size and/or aggregation can be selected to provide a glycoprotein preparation with a desired glycan property or properties. For example, normal phase liquid chromatography can be used to separate glycans and/or glycoproteins based on polarity. Reverse-phase chromatography can be used, e.g., with derivatized sugars. Anion-exchange columns can be used to purify sialylated, phosphorylated, and sulfated sugars. Other methods include high pH anion exchange chromatography and size exclusion chromatography can be used and is based on size separation.

Affinity based methods can be selected that preferentially bind certain chemical units and glycan structures. Matrices such as m-aminophenylboronic acid, immobilized lectins and antibodies can bind particular glycan structures. M-aminophenylboronic acid matrices can form a temporary covalent bond with any molecule (such as a carbohydrate) that contains a 1,2-cis-diol group. The covalent bond can be subsequently disrupted to elute the protein of interest. Lectins are a family of carbohydrate-recognizing proteins that exhibit affinities for various monosaccharides. Lectins bind carbohydrates specifically and reversibly. Primary monosaccharides recognized by lectins include mannose/glucose, galactose/N-acetylgalactosamine, N-acetylglucosamine, fucose, and sialic acid (QProteome Glycoarray Handbook, Qiagen, September 2005, available at: http://wolfson.huji.ac.il/purification/PDF/Lectins/QIAGEN_GlycoArrayHandbook.pdf) or similar references. Lectin matrices (e.g., columns or arrays) can consist of a number of lectins with varying and/or overlapping specificities to bind glycoproteins with specific glycan compositions. Some lectins commonly used to purify glycoproteins include concavalin A (often coupled to Sepharose or agarose) and Wheat Germ. Anti-glycan antibodies can also be generated by methods known in the art and used in affinity columns to bind and purify glycoproteins.

### Glycan Preparations

The present disclosure provides methods of analyzing the structure and/or composition of individual glycans within a glycan or glycoprotein preparation, e.g., evaluating glycans containing antennary fucose residues produced by CHO cells. A glycan preparation may be obtained from a cell preparation or from a glycoprotein preparation by any method available in the art. In general, obtaining a glycan preparation comprises steps of (1) obtaining a cell or glycoprotein preparation; and (2) optionally releasing glycans from the cell or glycoprotein preparation. In some embodiments, obtaining a glycan preparation optionally comprises labeling the glycan preparation with a detectable label.

In some embodiments, an N-glycan preparation is obtained by providing a glycoprotein population and removing N-linked glycans from the glycoproteins in the population. In some embodiments, N-linked glycans are removed from glycoproteins (e.g., glycoproteins) by digestion. Generally, glycanases to be used in accordance with the present disclosure cleave between GlcNAc-Asn, GlcNAc-GlcNAc, or Man-GlcNAc residues of the core. Exemplary enzymes which can be used to remove N-linked glycans from glycoproteins include, but are not limited to, N-glycanase F and/or N-glycanase-A, O-glycanase and/or Endo H. In some embodiments, N-linked glycans are removed from glycoproteins by chemical cleavage. To give but a few examples, hydrazine, sodium borohydride, and/or trifluoromethanesulfonic acid (TFMS) can be used to remove glycans from a glycoprotein.

### Labeling Glycans

In some embodiments, labels can be associated with glycans before or after release from a glycoprotein. N-linked glycans (e.g., N-glycans that have been removed from a glycoprotein population) can be associated with one or more detectable labels. Detectable labels are typically associated with the reducing ends of glycans. In some embodiments, detectable labels are fluorescent moieties. Exemplary fluorophores that can be used in accordance with the present disclosure include, but are not limited to, 2-aminobenzoic acid (2AA), 2-aminobenzamide (2AB), and/or 2-aminopurine (2AP). In general, fluorophores for use in accordance with the present disclosure are characterized by having reactivity with the reducing end of an oligosaccharide and/or monosaccharide under conditions that do not damage and/or destroy the glycan. In some embodiments, fluorescent moieties are attached to reducing ends directly. For example, direct attachment can be accomplished by direct conjugation by reductive amination. In some embodiments, fluorescent moieties are attached to reducing ends indirectly. For example, indirect attachment can be accomplished by a reactive linker arm.

In some embodiments, detectable labels comprise radioactive moieties or isotopically-labelled molecules. Exemplary radioactive moieties that can be used in accordance with the present disclosure include, but are not limited to, tritium (³H), deuterium (²H), and/or ³⁵S. Typically, such moieties are directly attached to or otherwise associated with the fluorophore. To give but one example of a radioactive fluorophore, 2AP can be modified such that all hydrogens are deuterated.

### Release of Glycans

The present disclosure provides improved methods of determining glycosylation patterns of glycoproteins. Such methods can involve subjecting a glycan population to one or more exoglycosidases and analyzing the structure and/or composition of the digestion products. In some embodiments, exoglycosidases used in accordance with the present disclosure recognize and cleave only one particular type of glycosidic linkage. In some embodiments, exoglycosidases used in accordance with the present disclosure recognize and cleave more than one particular type of glycosidic linkage. Among the exoglycosidases which may be useful for the present invention are α-galactosidases, β-galactosidases; hexosaminidases, mannosidases; and combinations thereof, as described in Table 1.

### Exoglycosidases

Exoglycosidases are enzymes that cleave terminal glycosidic bonds from the non-reducing end of glycans. They are typically highly specific to particular monosaccharide linkages and anomericity (α/β). In some embodiments, neighboring branching patterns can affect exoglycosidase specificity. Exoglycosidase treatment usually results in glycans of standard antennary linkages being cleaved down to the pentasaccharide core (M3N2) containing 3 mannose and 2 GlcNAc residues. However, unusually-modified species (e.g., antennary or core fucosylated species, high-mannose and hybrid glycans, lactosamine-extended glycans, sulfated glycans, phosphorylated glycans, etc.) are resistant to exoglycosidase treatment and can be chromatographically resolved and quantified relative to the M3N2 pentasaccharide.

Exemplary exoglycosidases that can be used in accordance with the present disclosure include, but are not limited to, sialidase, galactosidase, hexosaminidase, fucosidase, and mannosidase. Exoglycosidases can be obtained from any source, including commercial sources or by isolation and/or purification from a cellular source (e.g., bacteria, yeast, plant, etc.).

In some embodiments, exoglycosidases (e.g., sialidases, galactosidases, hexosaminidases, fucosidases, and mannosidases) can be divided into multiple categories or “subsets.” In some embodiments, the different subsets display different abilities to cleave different types of linkages. Table 1 presents some exemplary exoglycosidases, their linkage specificities, and the organism from which each is derived. One of ordinary skill in the art will appreciate that this is an exemplary, not a comprehensive, list of exoglycosidases, and that any exoglycosidase having any linkage specificity may be used in accordance with the present disclosure.

In some embodiments, antennary fucosylation can be detected and analyzed by using an α-1-3,4-fucosidase (e.g., an α-1-3,4-fucosidase described herein). For example, fucose residues attached to glycan antennae can be released by an α-1-3,4-fucosidase and the released monosaccharide and/or the remaining glycan antennae can be analyzed (e.g., quantified) by routine methods, e.g., HPLC or mass spectrometry.

TABLE 1
Exoglycosidases

| Enzyme class | EC #* | Activity | Organism |
|---|---|---|---|
| α-Sialidase | 3.2.1.18 | α-2/3,6,8 (usually not linkage- | Arthrobacter ureafaciens |
| | | specific) | Vibrio cholerae |
| | | | Clostridium perfringens |
| | | α-2,3 (NeuAc from oligosaccharides) | Salmonella typhimurium |
| | | | Streptococcus pneumonia |
| | | α-2/3,6 (NeuAc from complex) | Clostridium perfringens |
| β-Galactosidase | 3.2.1.23 | β-1/3,4,6 Gal linkages | Bovine testis |
| | | | Xanthamonas species |
| | | | Streptococcus species |
| | | | E. coli |
| | | β-1/4,6 Gal linkages | Jack bean |
| | | β-1,4 Gal linkage | Streptococcus pneumonia |
| | | β-1,3-Gal linkage | E. coli |
| | | | Xanthomonas species |
| | | β-1/3,6-Gal linkages | Xanthomonas species |
| | | | E. coli |
| β-Hexosaminidase | 3.2.1.52 | β-1/2,3,4,6 hexosamines | Streptococcus plicatus |
| | 3.2.1.30 | | Streptococcus pneumonia |
| | | | Bacteroides |
| | | | Jack bean |
| α-Fucosidase | 3.2.1.51 | α-1-3,4-Fuc (usually de-glycosylate | Xanthomonas |
| | 3.2.1.111 | Lewis structure) | Almond meal |
| | | α-1/2,3,4,6-Fuc (usually has broad | Bovine kidney |
| | | specificity) | C. meningosepticum |
| | | α-1,6-Fuc | E. coli |
| | | α-1,2-Fuc | Xanthomonas |
| α-Mannosidase | 3.2.1.24 | α-1/2,3,6-Man | Jack bean |
| | | α-1/2,3-Man | Xanthomonas manihotis |
| | | α-1,6-Man (typically a core | Xanthomonas species |
| | | mannosidase) | |
| | | α-1,2-Man | Aspergillus saitoi |
| β-Mannosidase | 3.2.1.25 | α-1,4-Man | Helix pomatia |

*“EC #” refers to Enzyme Commission registration number

According to the present disclosure, a glycan population can be digested with any exoglycosidase or any set of exoglycosidases. In general, exoglycosidase reactions take place under conditions that are compatible with enzyme activity. For example, pH, temperature, reaction solution components and concentration (e.g., salt, detergent, etc.), and length of reaction time can be optimized in order to achieve a desired level of exoglycosidase activity. See, e.g., WO 2008/130926, the contents of which are herein incorporated by reference.

### Analysis of Glycan Structure and Activity

In general, methods in accordance with the disclosure comprise subjecting a glycan preparation to analysis to determine whether glycans in the glycan preparation include a particular type of modification (e.g., an antennary fucose in addition to core fucose present on N-linked glycans). In some embodiments, the analysis comprises comparing the structure and/or function of glycans in one glycoprotein preparation from one source to structure and/or function of glycans in at least one other glycoprotein preparation from another source. In some embodiments, the analysis comprises comparing the structure and/or function of glycans in one or more of the samples to structure and/or function of glycans in a reference sample.

In some embodiments, glycans containing antennary fucosylation can be measured on a glycoprotein (or glycopeptides derived from the glycoprotein) without prior need of deglycosylation. For example glycopeptides containing antennary fucosylation may be measured using LC-MS/MS, MRM, HPLC, HPLC, tandem MS techniques as described in the literature. Antennary fucosylated glycans can also be measured directly on the glycoprotein using techniques such as CE-MS and HPLC after exoglycosidase treatment.

Structure and composition of glycans can be analyzed by any available method. In some embodiments, glycan structure and composition as described herein are analyzed by chromatographic methods, mass spectrometry (MS) methods, chromatographic methods followed by MS, electrophoretic methods, electrophoretic methods followed by MS, nuclear magnetic resonance (NMR) methods, and combinations thereof.

In some embodiments, glycan structure and composition can be analyzed by chromatographic methods, including but not limited to, high performance anion-exchange chromatography with pulsed amperometric detection (HPAE-PAD), liquid chromatography (LC), high performance liquid chromatography (HPLC), ultra performance liquid chromatography (HPLC), thin layer chromatography (TLC), amide column chromatography, and combinations thereof.

In some embodiments, glycan structure and composition can be analyzed by mass spectrometry (MS) and related methods, including but not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof.

In some embodiments, glycan structure and composition can be analyzed by electrophoretic methods, including but not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof.

In some embodiments, glycan structure and composition can be analyzed by nuclear magnetic resonance (NMR) and related methods, including but not limited to, one-dimensional NMR (1D-NMR), two-dimensional NMR (2D-NMR), correlation spectroscopy magnetic-angle spinning NMR(COSY-NMR), total correlated spectroscopy NMR (TOCSY-NMR), heteronuclear single-quantum coherence NMR(HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

In some embodiments, techniques described herein may be combined with one or more other technologies for the detection, analysis, and or isolation of glycans or glycoproteins. For example, in certain embodiments, glycans are analyzed in accordance with the present disclosure using one or more available methods (to give but a few examples, see Anumula, Anal. Biochem. 350(1):1, 2006; Klein et al., Anal. Biochem., 179:162, 1989; and/or Townsend, R. R. Carbohydrate Analysis” High Performance Liquid Chromatography and Capillary Electrophoresis., Ed. Z. El Rassi, pp 181-209, 1995, each of which is incorporated herein by reference in its entirety). For example, in some embodiments, glycans are characterized using one or more of chromatographic methods, electrophoretic methods, nuclear magnetic resonance methods, and combinations thereof. Exemplary such methods include, for example, NMR, mass spectrometry, liquid chromatography, 2-dimensional chromatography, SDS-PAGE, antibody staining, lectin staining, monosaccharide quantitation, capillary electrophoresis, fluorophore-assisted carbohydrate electrophoresis (FACE), micellar electrokinetic chromatography (MEKC), exoglycosidase or endoglycosidase treatments, and combinations thereof. Those of ordinary skill in the art will be aware of other techniques that can be used to characterize glycans together with the methods described herein.

In some embodiments, methods described herein allow for detection of glycan species or particular structures (such as antennary fucose-containing glycans) that are present at low levels within a population of glycans. For example, the present methods allow for detection of glycan species that are present at levels less than 40%, 30%, 25%, 20%, 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, methods described herein allow for detection of relative levels of individual glycan species within a population of glycans. For example, the area under each peak of a liquid chromatograph can be measured and expressed as a percentage of the total. Such an analysis provides a relative percent amount of each glycan species within a population of glycans. In another example, relative levels of individual glycan species are determined from areas of peaks in a 1D-NMR experiment, or from volumes of cross peaks from a 1H-15HSQC spectrum (e.g., with correction based on responses from standards), or by relative quantitation by comparing the same peak across samples.

In some embodiments, a biological activity of a glycoprotein preparation (e.g., a glycoprotein preparation) is assessed. Biological activity of glycoprotein preparations can be analyzed by any available method. In some embodiments, a binding activity of a glycoprotein is assessed (e.g., binding to a receptor). In some embodiments, a therapeutic activity of a glycoprotein is assessed (e.g., an activity of a glycoprotein in decreasing severity or symptom of a disease or condition, or in delaying appearance of a symptom of a disease or condition). In some embodiments, a pharmacologic activity of a glycoprotein is assessed (e.g., bioavailability, pharmacokinetics, pharmacodynamics). For methods of analyzing bioavailability, pharmacokinetics, and pharmacodynamics of glycoprotein therapeutics, see, e.g., Weiner et al., J Pharm Biomed Anal. 15(5):571-9, 1997; Srinivas et al., J. Pharm. Sci. 85(1):1-4, 1996; and Srinivas et al., Pharm. Res. 14(7):911-6, 1997.

As would be understood to one of skill in the art, the particular biological activity or therapeutic activity that can be tested will vary depending on the particular glycoprotein.

The potential adverse activity or toxicity (e.g., propensity to cause hypertension, allergic reactions, thrombotic events, seizures, or other adverse events) of glycoprotein preparations can be analyzed by any available method. In some embodiments, immunogenicity of a glycoprotein preparation is assessed, e.g., by determining whether the preparation elicits an antibody response in a subject, such as an experimental animal.

In various embodiments, biological activity, therapeutic activity, etc., of a glycoprotein preparation having antennary fucose residues is compared to a glycoprotein preparation lacking antennary fucose residues residues. In various embodiments, biological activity, therapeutic activity, etc., of a glycoprotein preparation having antennary fucose residues is compared to a glycoprotein preparation having a different level of antennary fucose residues.

### Applications

Methods of the present disclosure can be utilized to analyze glycans in any of a variety of states including, for instance, free glycans, glycoproteins (e.g., glycopeptides, glycolipids, proteoglycans, etc.), cell-associated glycans (e.g., nucleus-, cytoplasm-, cell-membrane-associated glycans, etc.); glycans associated with cellular, extracellular, intracellular, and/or subcellular components (e.g., proteins); glycans in extracellular space (e.g., cell culture medium), etc.

Methods of the present disclosure may also be used in one or more stages of process development for the production of a therapeutic or other commercially relevant glycoprotein. Non-limiting examples of stages that can employ methods of the present disclosure include cell selection, clonal selection, media optimization, assessment of culture conditions, process conditions, and/or purification procedure. Those of ordinary skill in the art will be aware of other process development stages.

Compositions and methods described herein are also useful during commercial production of therapeutic glycoproteins. For example, during cell culture (e.g., in a commercial bioreactor) of appropriate recombinant cells, such as CHO host cells genetically engineered to produce a therapeutic glycoprotein, during polypeptide purification steps, during drug product formulation, and as part of testing of a drug substance or drug product. Methods of producing therapeutic glycoproteins as described herein will employ techniques known in the art, e.g., as described in Current Protocols in Molecular Biology (2007, John Wiley and Sons, Inc., Print ISSN: 1934-3639); Current Protocols in Cell Biology (2007, John Wiley and Sons, Inc., Print ISSN: 1934-2500); Current Protocols in Protein Science (2007, John Wiley and Sons, Inc., Print ISSN: 1934-3655): Wurm, Production of recombinant protein therapeutics in cultivated mammalian cells (2004) Nature Biotech. 22:1393-1398; Therapeutic Proteins: Methods and Protocols, Smales and James, eds. (2005, Humana Press, ISBN-10: 1588293904).

The present disclosure can also be utilized to monitor the extent and/or type of glycosylation occurring in a particular cell culture (e.g., the level of antennary fucose residues in a glycoprotein preparation produced in the cell culture), thereby allowing adjustment or possibly termination of the culture in order, for example, to achieve a particular desired glycosylation pattern or to avoid development of a particular undesired glycosylation pattern.

The present disclosure can also be utilized to assess glycosylation characteristics of cells or cell lines (e.g., CHO cell lines) that are being considered for production of a particular desired glycoprotein (for example, even before the cells or cell lines have been engineered to produce the glycoprotein, or to produce the glycoprotein at a commercially relevant level).

For example, where the target glycoprotein is a therapeutic glycoprotein, for example having undergone regulatory review in one or more countries, it will often be desirable to monitor cultures to assess the likelihood that they will generate a product with a glycosylation pattern as close to the established glycosylation pattern of the pharmaceutical product as possible (e.g., having a degree of antennary fucosylation which is close to that of the pharmaceutical product), whether or not it is being produced by exactly the same route. As used herein, “close” means within a predetermined acceptable range. For example, “close” may refer to a glycosylation pattern having at least about a 75%, 80%, 85%, 90%, 95%, 98%, or 99% correlation to the established glycosylation pattern of the pharmaceutical product. In some embodiments, “close” may refer to a glycosylation pattern that lacks or contains one or more particular structure(s), or includes such structures at a level that is within a predetermined range or a predetermined relationship to a threshold value. In such embodiments, samples of the production culture are typically taken at multiple time points and are compared with an established standard or with a control culture in order to assess relative glycosylation.

For example, in some embodiments, methods for monitoring production of a glycoprotein may comprise steps of (i) during production of a glycoprotein, removing at least first and second glycan-containing samples from the production system; (ii) subjecting each of the first and second glycan-containing samples to an analysis to determine whether a particular modification is present (e.g., antennary fucosylation); and (iii) comparing the products obtained from the first glycan-containing sample with those obtained from the second glycan-containing sample so that differences are determined and therefore progress of glycoprotein production is monitored. In some embodiments, the glycoprotein is a therapeutic antibody. In certain embodiments, the production system comprises CHO cells.

Whether or not monitoring production of a particular target protein for quality control purposes, the present disclosure may be utilized, for example, to monitor glycosylation at particular stages of development, or under particular growth conditions.

In some embodiments, methods described herein can be used to characterize, modulate and/or control or compare the quality of therapeutic products. To give but one example, the present methodologies can be used to assess glycosylation in cells producing a therapeutic protein product. Particularly given that glycosylation can often affect the activity, bioavailability, or other characteristics of a therapeutic protein product, methods for assessing cellular glycosylation during production of such a therapeutic protein product are particularly desirable. Among other things, the present disclosure can facilitate real time analysis of glycosylation in production systems for therapeutic proteins, and hence, modulation of the glycosylation may be achieved.

Representative therapeutic glycoprotein products whose production and/or quality can be monitored in accordance with the present disclosure include, for example, any of a variety of hematologic agents (including, for instance, erythropoietin, blood-clotting factors, etc.), interferons, colony stimulating factors, therapeutic antibodies, enzymes, and hormones.

Representative commercially available glycoprotein products include, for example, those presented in Table 2, if produced in CHO cells.

TABLE 2
Exemplary commercially available glycoprotein products

| Protein Product | Reference Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| laronidase | Aldurazyme ® |
| interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion protein LFA3-Ig | Amevive ® |
| bivalirudin | Angiomax ® |
| darbepoetin alfa | Aranesp ™ |
| bevacizumab | Avastin ™ |
| interferon beta-1a; recombinant | Avonex ® |
| coagulation factor IX | BeneFix ™ |
| Interferon beta-1b | Betaseron ® |
| Tositumomab | Bexxar ® |
| antihemophilic factor | Bioclate ™ |
| human growth hormone | BioTropin ™ |
| botulinum toxin type A | Botox ® |
| alemtuzumab | Campath ® |
| acritumomab; technetium-99 labeled | CEA-Scan ® |
| alglucerase; modified form of beta-glucocerebrosidase | Ceredase ® |
| imiglucerase | Cerezyme ® |
| crotalidae polyvalent immune Fab, ovine | CroFab ™ |
| digoxin immune Fab, ovine | DigiFab ™ |
| rasburicase | Elitek ® |
| etanercept | Enbrel ® |
| epoietin alfa | Epogen ® |
| cetuximab | Erbitux ™ |
| algasidase beta | Fabrazyme ® |
| urofollitropin | Fertinex ™ |
| follitropin beta | Follistim ™ |
| teriparatide | Forteo ® |
| human somatropin | GenoTropin ® |
| glucagon | GlucaGen ® |
| follitropin alfa | Gonal-F ® |
| antihemophilic factor | Helixate ® |
| Antihemophilic Factor; Factor XIII | Hemofil ® |
| insulin | Humalog ® |
| antihemophilic factor/von Willebrand factor complex | Humate-P ® |
| somatotropin | Humatrope ® |
| adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| palifermin | Kepivance |
| anakinra | Kineret ™ |
| antihemophilic factor | Kogenate ®FS |
| insulin glargine | Lantus ® |
| granulocyte macrophage colony-stimulating factor | Leukine ®/Leukine ® Liquid |
| lutropin alfa, for injection | Luveris |
| OspA lipoprotein | LYMErix ™ |
| ranibizumab | Lucentis ® |
| gemtuzumab ozogamicin | Mylotarg ™ |
| galsulfase | Naglazyme ™ |
| nesiritide | Natrecor ® |
| pegfilgrastim | Neulasta ™ |
| oprelvekin | Neumega ® |
| filgrastim | Neupogen ® |
| fanolesomab | NeutroSpec ™ (formerly LeuTech ®) |
| somatropin [rDNA] | Norditropin ®/Norditropin Nordiflex ® |
| insulin; zinc suspension; | Novolin L ® |
| insulin; isophane suspension | Novolin N ® |
| insulin, regular; | Novolin R ® |
| insulin | Novolin ® |
| coagulation factor VIIa | NovoSeven ® |
| somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| human chorionic gonadotropin | Ovidrel ® |
| peginterferon alfa-2a | Pegasys ® |
| pegylated version of interferon alfa-2b | PEG-Intron ™ |
| Abarelix; gonadotropin-releasing hormone antagonist | Plenaxis ™ |
| epoietin alfa | Procrit ® |
| aldesleukin | Proleukin, IL-2 ® |
| somatrem | Protropin ® |
| dornase alfa | Pulmozyme ® |
| Efalizumab; selective, reversible T-cell blocker | Raptiva ™ |
| combination of ribavirin and alpha interferon | Rebetron ™ |
| Interferon beta 1a | Rebif ® |
| antihemophilic factor | Recombinate ® |
| rAHF/ntihemophilic factor | ReFacto ® |
| lepirudin | Refludan ® |
| infliximab | Remicade ® |
| abciximab | ReoPro ™ |
| reteplase | Retavase ™ |
| rituximab | Rituxan ™ |
| interferon alfa-2a | Roferon-A ® |
| somatropin | Saizen ® |
| synthetic porcine secretin | SecreFlo ™ |
| basiliximab | Simulect ® |
| eculizumab | Soliris ® |
| pegvisomant | Somavert ® |
| Palivizumab; recombinantly produced, humanized mAb | Synagis ™ |
| thyrotropin alfa | Thyrogen ® |
| tenecteplase | TNKase ™ |
| natalizumab | Tysabri ® |
| human immune globulin intravenous | Venoglobulin-S ® |
| interferon alfa-n1, lymphoblastoid | Wellferon ® |
| drotrecogin alfa | Xigris ™ |
| Omalizumab | Xolair ® |
| daclizumab | Zenapax ® |
| ibritumomab tiuxetan | Zevalin ™ |
| Somatotropin | Zorbtive ™ (Serostim ®) |
| denosumab | Prolia ® |
| panitumumab | Vectibi ® |

Information about the amino acid sequence of the recombinant products listed in Table 2 can be found, e.g., in product literature, e.g., in the Prescribing Information for the relevant product. Alternatively, methods of sequencing proteins to obtain the amino acid sequence of a glycoprotein drug product are known in the art. “Percent (%) sequence identity” with respect to a sequence is defined as the percentage of amino acid residues or nucleotides in a candidate sequence that are identical with the amino acid residues or nucleotides in the reference sequence, after aligning the sequences and introducing gaps, if necessary, to achieve the maximum percent sequence identity. (E.g., gaps can be introduced in one or both of a first and a second amino acid or nucleic acid sequence for optimal alignment and non-homologous sequences can be disregarded for comparison purposes). Alignment for purposes of determining percent sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. In one embodiment, the length of a reference sequence aligned for comparison purposes is at least 30%, e.g., at least 40%, e.g., at least 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

In some embodiments, the disclosure provides methods in which glycans from glycoproteins from different sources or samples are compared with one another. In some such examples, multiple samples from the same source (e.g., from the same CHO cell source) are obtained over time, so that changes in glycosylation patterns (and particularly in cell surface glycosylation patterns) (e.g., changes in the presence or extent of antennary fucose residues) are monitored. In some embodiments, one of the samples is a historical sample or a record of a historical sample. In some embodiments, one of the samples is a reference sample.

In some embodiments, the disclosure provides methods in which glycans from glycoproteins expressed by different cell sources are compared with one another. In some embodiments, one or more of the compared cell sources are different populations of CHO cells.

In some embodiments, glycans from different cell culture samples prepared under conditions that differ in one or more selected parameters (e.g., cell type, culture type [e.g., continuous feed vs. batch feed, etc.], culture conditions [e.g., type of media, presence or concentration of particular component of particular medium(a), osmolarity, pH, temperature, timing or degree of shift in one or more components such as osmolarity, pH, temperature, etc.], culture time, isolation steps, etc.) but are otherwise identical, are compared, so that effects of the selected parameter on glycosylation are determined. In certain embodiments, glycans from different cell culture samples prepared under conditions that differ in a single selected parameter are compared so that effects of the single selected parameter on glycosylation patterns (e.g., the presence or absence of antennary fucose residues) are determined. Among other applications, therefore, use of techniques as described herein may facilitate determination of the effects of particular parameters on glycosylation patterns in cells.

In some embodiments, glycans from different batches of a glycoprotein, whether prepared by the same method or by different methods, and whether prepared simultaneously or separately, are compared. In such embodiments, the present disclosure facilitates quality control of a glycoprotein preparation. Alternatively or additionally, some such embodiments facilitate monitoring of progress of a particular culture producing a glycoprotein (e.g., when samples are removed from the culture at different time points and are analyzed and compared to one another). In some examples, multiple samples from the same source are obtained over time, so that changes in glycosylation patterns are monitored. In some embodiments, glycan-containing samples are removed at about 30 second, about 1 minute, about 2 minute, about 5 minute, about 10 minute, about 30 minute, about 1 hour, about 2 hour, about 3 hour, about 4 hour, about 5 hour, about 10 hour, about 12 hour, or about 18 hour intervals, or at even longer intervals. In some embodiments, glycan-containing samples are removed at irregular intervals. In some embodiments, glycan-containing samples are removed at 5 hour intervals.

In some embodiments, methods in accordance with the disclosure may be used to monitor the glycosylation pattern of glycoproteins during the course of their production by cells. For example, production of a glycoprotein (e.g., commercial production) may involve steps of (1) culturing cells that produce the glycoprotein, (2) obtaining samples at regular or irregular intervals during the culturing, and (3) analyzing the glycosylation pattern of produced glycoprotein(s) in obtained sample(s). In some embodiments, such methods may comprise a step of comparing the glycosylation patterns of produced glycoprotein(s) in obtained samples to one another. In some embodiments, such methods may comprise a step of comparing glycosylation patterns of produced glycoprotein(s) in obtained sample(s) to the glycosylation pattern of a reference sample.

In any of these embodiments, features of the glycan analysis described herein can be recorded, for example in a print or electronic record, e.g., a Material Safety Data Sheet (MSDS) or Certificate of Testing or Certificate of Analysis (CofA). As indicated above, in some embodiments, a comparison is with a historical record of a prior or standard batch and/or with a reference sample of glycoprotein.

In some embodiments, glycans from different batches of a particular glycoprotein, whether prepared by the same method or by different methods, and whether prepared simultaneously or separately, are compared to one another and/or to a reference sample. In some embodiments, batch-to-batch comparison may comprise the steps of (i) providing a first glycan preparation from a first batch of the glycoprotein; (ii) providing a second glycan preparation from a second batch of the glycoprotein; (iii) subjecting each of the first and second glycan preparations to analysis procedure; and (iv) comparing the results of the analysis obtained from the first glycan preparation with the cleavage products obtained from the second preparation so that consistency of the two batches is assessed. In some embodiments, glycan preparations can be provided by removing at least one glycan from at least one glycoprotein from a batch and, optionally, isolating removed glycans. In some embodiments, glycan preparations may be labeled as described herein (e.g., fluorescently and/or radioactively; e.g., prior to and/or after isolation).

In some embodiments, the present disclosure facilitates quality control of a glycoprotein preparation. Features of the glycan analysis can be recorded, for example in a quality control record. As indicated above, in some embodiments, a comparison is with a historical record of a prior or standard batch of glycoprotein. In some embodiments, a comparison is with a reference glycoprotein sample.

In certain embodiments, the present disclosure may be utilized in studies to modify the glycosylation characteristics of a cell, for example to establish a cell line and/or culture conditions with one or more desirable glycosylation characteristics, e.g., a cell line that produces glycoproteins having, or lacking, antennary fucose. Such a cell line and/or culture conditions can then be utilized, if desired, for production of a particular target glycoprotein for which such glycosylation characteristic(s) is/are expected to be beneficial. In particular embodiments, the cell is a CHO cell.

According to the present disclosure, techniques described herein can be used to detect desirable or undesirable glycans, for example to detect or quantify the presence of one or more contaminants in a glycoprotein product, or to detect or quantify the presence of one or more active or desired species.

In certain embodiments, methods described herein facilitate detection of glycan species that are present at very low levels in a source (e.g., a biological sample, glycan preparation, etc.). In such embodiments, it is possible to detect and/or optionally quantify the levels of glycans that are present at levels less than about 20%, 10%, 5%, 4%, 3%, 2%, 1.5%, 1%, 0.75%, 0.5%, 0.25%, 0.1%, 0.075%, 0.05%, 0.025%, or 0.01% within a population of glycans. In some embodiments, it is possible to detect and/or optionally quantify the levels of glycans comprising between 0.1% and 5%, e.g., between 0.1% and 2%, e.g., between 0.1% and 1% of a glycan preparation.

In some embodiments, methods described herein allow for detection of relative levels of individual glycan species within a population of glycans. For example, the area under each peak of a liquid chromatograph can be measured and expressed as a percentage of the total. Such an analysis provides a relative percent amount of each glycan species within a population of glycans.

The present disclosure will be more specifically illustrated with reference to the following examples. However, it should be understood that the present disclosure is not limited by these examples in any manner.

One of skill in the art may readily envision various other combinations within the scope of the present invention, considering the example with reference to the specification herein provided.

### EXAMPLES

### Example 1

### Identification of Glycan Containing Antennary Fucose in CHO

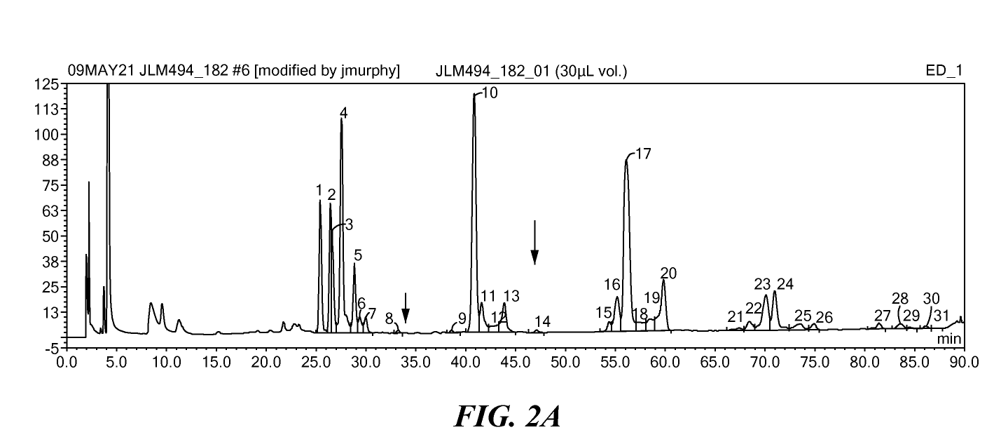

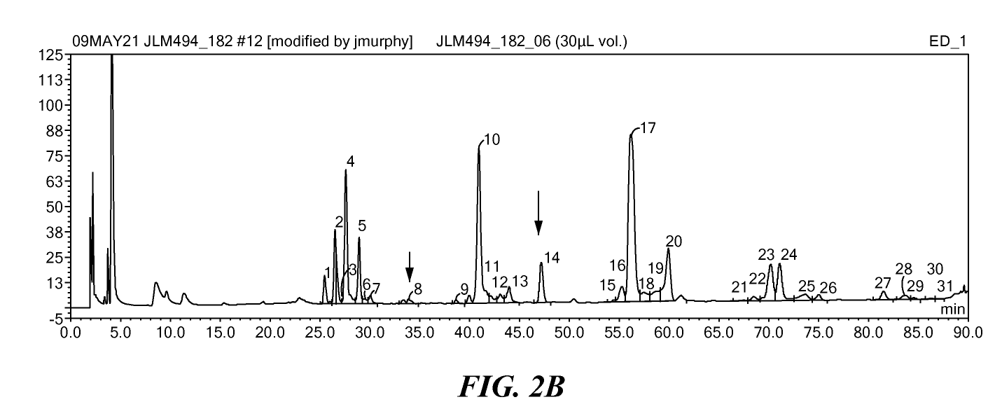

A recombinant Fc fusion protein coding sequence (CTLA4-Ig; see WO 2007/076032) was transfected into CHO-K1 cells, amplified with methotrexate and single clones isolated by dilution cloning. The individual clones were expanded and cultured for 5 days, prior to being harvested. The resultant media (supernatant) was clarified and the recombinant Fc fusion protein was purified by protein A affinity chromatography. The harvested cells were concurrently lysed for isolation of total RNA and subsequent transcriptional analysis by quantitative, real-time PCR (qPCR). Glycans were released from the purified glycoprotein with N-glycanase and purified by PGC chromatography. The glycans were then analyzed by high performance anion-exchange chromatography with pulsed amperometric detection (HPAE-PAD) as generally described in Hayase et al., Analytical Biochemistry, 1993, 211: 72-80. For this experiment, a gradient of 2-100% 250 mM ammonium acetate in 86 minutes was used as the eluting solvent along with 100 mM sodium hydroxide. A typical profile is shown in FIG. 2A.

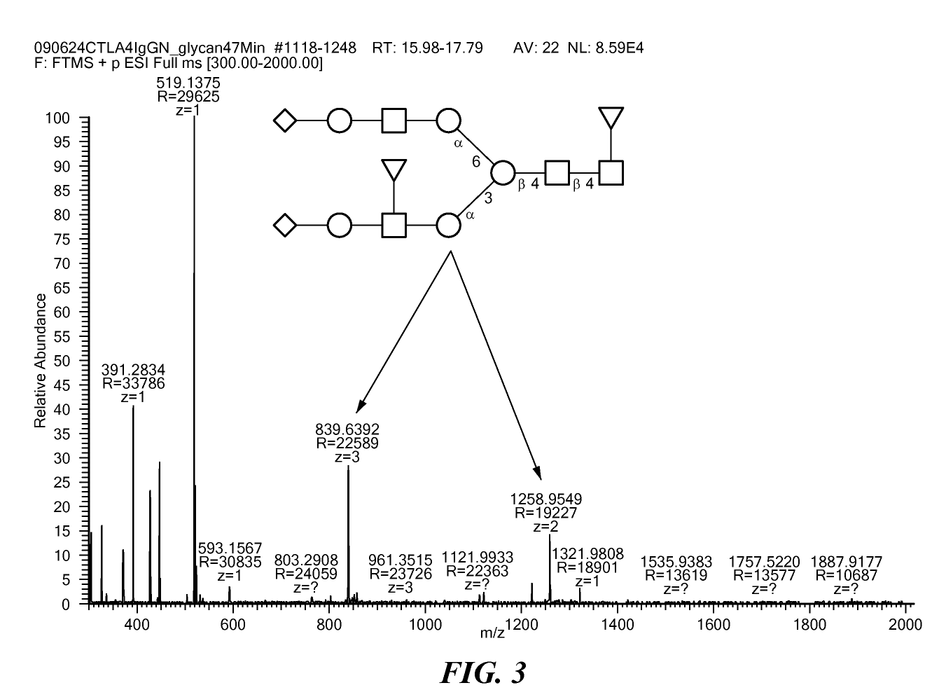

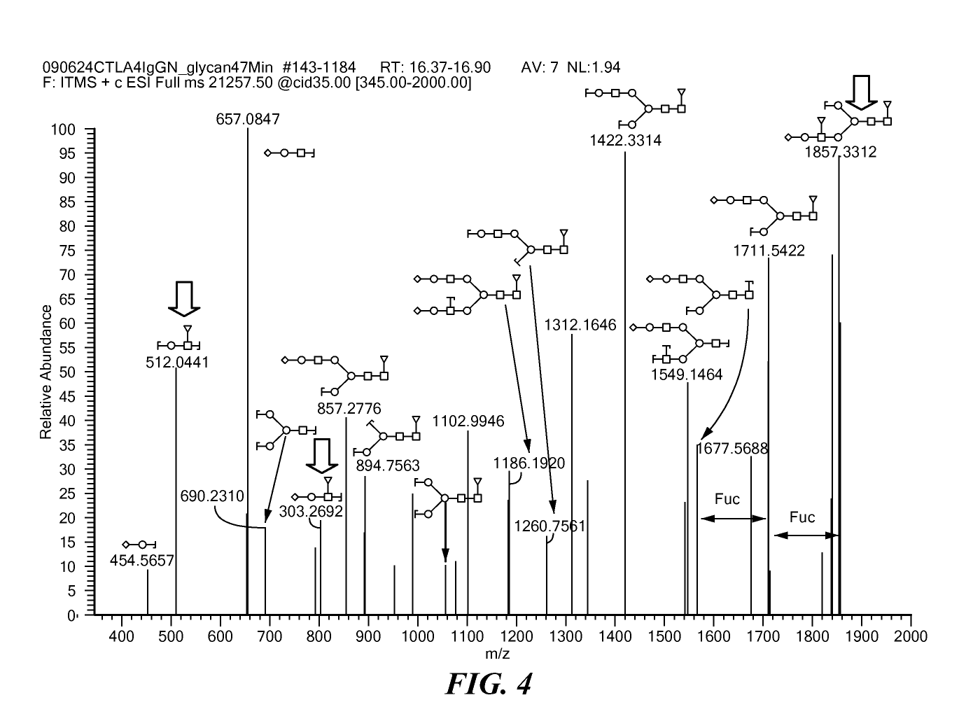

Among multiple clones analyzed as described above, some clones showed two additional, unknown peaks: a small peak eluting between the neutral and monosialylated fractions (around 34 minutes) and a larger peak eluting between the monosialylated and disialylated fractions (around 47 minutes), highlighted by the smaller and longer arrow, respectively, in the profile of FIG. 2B, and missing in the typical profile of FIG. 2A. These peaks were further analyzed via mass spectrometry and exoglycosidase analysis to identify that they contain antennary fucosylation. For example, the mass spectrometry analysis of the atypical peak eluting around 47 minutes indicated to be a single glycan species with a molecular weight of 2515 Da (shown in FIG. 3). Further analysis of this peak by MS-MS and exoglycosidase cleavage revealed the structure of this glycan species to be a bifucosylated glycan with an antennary fucose in addition to core fucose. The fucosylated fragments resulting from the MS/MS analysis highlighted in FIG. 4 (block arrow) shows the presence of the antennary fucose on the glycan species.

### Example 2

### Screening for Antennary Fucosylation in CHO Cells

The above identified antennary fucosylated species were monitored during the screening of clones from different CHO cell lines using the same methodology as in Example 1.

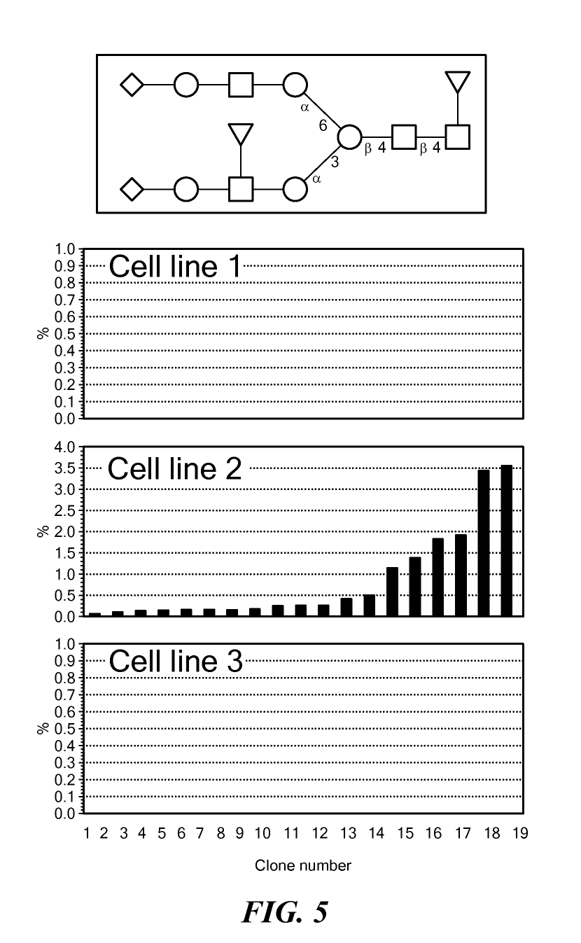

As shown in FIG. 5, this method was easily able to distinguish cell lines that produce antennary/bifucosylated glycans (e.g., cell line 2: CHO-K1) from cell line clones that do not (e.g., cell lines 1 and 3: CHO-DG44 and CHO-S clones), and to quantify very low levels of bifucosylated glycan species in different clones. For example, this method was able to detect bifucosylated species present as about 0.05% and 0.1%, respectively, of the total glycan pool (see FIG. 5, clones 1 and 2 of cell line 2). Accordingly, this method allows sensitive, rapid and high throughput identification and quantitation of antennary fucosylated glycans.

### Example 3

### Production of Glycoproteins Having Altered Antennary Fucosylation

Multiple clones of CHO-K1 cells were used as host cells to produce recombinant CTLA4-Ig. Glycans from the resulting products were analyzed by HPAE-PAD as described above. As shown in FIG. 5 (middle panel) CTLA4-Ig having varying or altered levels of antennary fucosylation were produced by different clones. Accordingly, the parent CHO cell clones provide useful reagents for expression of recombinant glycoproteins having targeted levels of branched fucose.

### Extensions and Alternatives

While the methods have been particularly shown and described with reference to specific illustrative embodiments, it should be understood that various changes in form and detail may be made without departing from the spirit and scope of the present disclosure. Therefore, all embodiments that come within the scope and spirit of the methods, and equivalents thereto, are intended to be claimed. The claims, descriptions and diagrams of the methods, systems, and assays of the present disclosure should not be read as limited to the described order of elements unless stated to that effect.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way. While the methods have been described in conjunction with various embodiments and examples, it is not intended that the methods be limited to such embodiments or examples. On the contrary, the methods encompass various alternatives, modifications, and equivalents, as will be appreciated by those of skill in the art.

We claim:

1. A method for evaluating a Chinese Hamster Ovary (CHO) cell population, the method comprising:
(a) providing one or more CHO cells from the population; and
(b) measuring glycans containing antennary fucose residues produced by said cells, wherein the CHO cells have not been genetically engineered to express an α3/α4 antennary fucosyltransferase coding sequence.

2. The method of claim 1, wherein the measuring step comprises any of:
(a) isolating glycoproteins produced from the CHO cells and measuring the glycans containing antennary fucose residues on the glycoproteins,
(b) isolating a specific glycoprotein composition produced from the CHO cells and measuring the glycans containing antennary fucose residues from the isolated glycoprotein composition,
(c) obtaining a glycan preparation from a glycoprotein preparation or isolated glycoprotein produced from the CHO cells and measuring the glycans containing antennary fucose residues in the glycan preparation,
(d) cleaving the antennary fucose monosaccharides from glycans present on a glycoprotein produced from the CHO cells or from glycans on the surface of the one or more CHO cells, and detecting the cleaved fucose monosaccharides,
(e) providing at least one peptide from a glycoprotein preparation produced from the CHO cells, and measuring the glycans containing antennary fucose residues on the at least one peptide,
(f) measuring glycans containing antennary fucose residues from glycans on the cell surface of the one or more CHO cells, and
(g) measuring expression of one or more FucT I, II, III, IV, V, VI, VII, or IX gene in the cells.

3. The method of claim 1, wherein the CHO cell population is a clonal cell population.

4. The method of claim 1, further comprising a step of comparing the level measured in step (b) to a reference level or specification.

5. The method of claim 4, wherein the reference level or specification is the level of antennary fucosylation found in a reference pharmaceutical product.

6. The method of claim 1, wherein measuring comprises use of a method for identifying or quantifying glycans containing antennary fucose residues selected from the group consisting of: chromatographic methods, mass spectrometry (MS) methods, electrophoretic methods, nuclear magnetic resonance (NMR) methods, monosaccharide analysis, fluorescence methods, UV-VIS absorbance, enzymatic methods, use of a detection molecule, and combinations thereof.

7. The method of claim 1, wherein measuring comprises performing High Performance anion-exchange chromatography with pulsed amperometric detection (HPAE-PAD).

8. The method of claim 1, wherein measuring comprises LC-MS or tandem MS.

9. The method of claim 1, wherein the CHO cell population is in a non-commercial bioreactor.

10. The method of claim 1, wherein the CHO cell population is in a commercial bioreactor.

11. The method of claim 1, wherein the CHO cells in the population have been transformed with a vector encoding a human therapeutic glycoprotein.

12. The method of claim 1, wherein the providing and measuring steps are repeated at least once over time.

13. The method of claim 1, further comprising a step of recording the result of the measuring step in a print or electronic record.

14. The method of claim 13, wherein the print or electronic record is a test report, a Certificate of Testing, a Certificate of Analysis, or a Material Safety Data Sheet.

15. The method of claim 1, further comprising the step of quantifying the amount of antennary fucose residues or glycans containing the residues.

16. The method of claim 1, wherein the measuring step comprises isolating glycoproteins produced from the CHO cells and measuring the glycans containing antennary fucose residues on the glycoproteins.

17. The method of claim 1, wherein measuring comprises isolating a specific glycoprotein composition produced from the CHO cells and measuring the glycans containing antennary fucose residues from the isolated glycoprotein composition.

18. The method of claim 1, wherein measuring comprises obtaining a glycan preparation from a glycoprotein preparation or isolated glycoprotein produced from the CHO cells and measuring the glycans containing antennary fucose residues in the glycan preparation.

19. The method of claim 1, wherein the measuring comprises cleaving monosaccharides from glycans present on a glycoprotein produced from the CHO cells or from glycans on the surface of the one or more CHO cells, and detecting the antennary fucose residues.

20. The method of claim 1, wherein measuring comprises providing at least one peptide from a glycoprotein preparation produced from the CHO cells, and measuring the glycans containing antennary fucose residues on the at least one peptide.

21. The method of claim 1, wherein measuring comprises measuring antennary fucose residues on glycoconjugates on the cell surface of the one or more CHO cells.

22. The method of claim 1, wherein the measuring step comprises performing a chromatographic method.

23. The method of claim 1, wherein the measuring step comprises performing a mass spectrometry (MS) method.

24. The method of claim 1, wherein the measuring step comprises performing an electrophoretic method.

25. The method of claim 1, wherein the measuring step comprises performing a nuclear magnetic resonance (NMR) method.

26. A method for screening one or more Chinese Hamster Ovary (CHO) cells for the ability to produce glycoproteins comprising glycans containing antennary fucose, the method comprising:
(a) providing a plurality of CHO cell populations wherein none of the plurality have been genetically engineered to produce antennary fucose residues on glycans;
(b) culturing each of the plurality of CHO cell populations under conditions suitable for expression of a glycoprotein expression product;
(c) measuring glycans containing antennary fucose residues produced by each of the plurality of CHO cells, and
(d) selecting one or more of the plurality of CHO cell preparations based on the presence of a target level of antennary fucose residues produced by the selected CHO cell preparation, wherein the CHO cells have not been transfected with an α3/α4 antennary fucosyltransferase coding sequence.

27. The method of claim 26, wherein the target level of antennary fucose is the level of antennary fucosylation found in a reference glycoprotein pharmaceutical product.

28. The method of claim 26, wherein the glycans containing term antennary fucose residues are measured on an isolated glycoprotein expression product of the CHO cell preparations.

29. The method of claim 26, wherein the glycans containing antennary fucose residues are measured on peptides obtained from a glycoprotein expression product of the CHO cell preparations.

30. The method of claim 26, wherein the glycans containing antennary fucose residues are measured from cell surface glycans of the CHO cell preparations.

31. The method of claim 26, wherein the glycans containing antennary fucose residues are measured on glycan preparations obtained from the CHO cell preparations or from a glycoprotein expression product thereof.

32. The method of claim 26, wherein the measuring step includes the step of (i) isolating a glycoprotein expression product from each of the plurality of CHO cell populations, and (ii) measuring the antennary fucose residues on the glycoprotein expression product.

33. The method of claim 27, wherein the cell culture is in a bioreactor.

34. The method of claim 26, wherein measuring comprises use of a technique for identifying or quantifying glycans containing antennary fucose residues selected from the group consisting of: chromatographic methods, mass spectrometry (MS) methods, electrophoretic methods, nuclear magnetic resonance (NMR) methods, monosaccharide analysis, fluorescence methods, UV-VIS absorbance, enzymatic methods, use of a detection molecule, and combinations thereof.

35. The method of claim 26, wherein at least one the plurality of CHO cell populations have been transformed with a vector encoding a human therapeutic glycoprotein.

36. The method of claim 26, wherein the plurality of CHO cell populations comprises at least one characteristic selected from the group consisting of: at least two different CHO strains, at least two different clonal cell populations, and at least two different samples from a manufacturing process train for a therapeutic glycoprotein.

37. The method of claim 26, further comprising the step of culturing the selected CHO cell preparation to produce a therapeutic glycoprotein product.

38. A method for evaluating a glycoprotein composition produced in a CHO cell host, comprising: measuring the amount of antennary fucose present in a glycoprotein composition, wherein the glycoprotein composition was produced in CHO host cells, and wherein the CHO host cells were not genetically engineered to express an α3/α4 antennary fucosyltransferase coding sequence.

39. The method of claim 38, further comprising recording the level of antennary fucose present in the glycoprotein composition in a print or computer-readable record.

40. The method of claim 38, further comprising comparing the measured level of antennary fucose present in the glycoprotein composition with a reference level.

41. The method of claim 40, wherein the reference level is a level of antennary fucosylation found in a reference pharmaceutical product specification.

42. The method of claim 40, wherein the reference level is no more than 20% antennary fucose on a glycan/total glycan basis.

43. The method of claim 38, wherein measuring the amount of antennary fucose present in the glycoprotein composition comprises performing a chromatographic method.

44. The method of claim 38, wherein measuring the amount of antennary fucose present in the glycoprotein composition comprises performing a mass spectrometry (MS) method.

45. The method of claim 38, wherein measuring the amount of antennary fucose present in the glycoprotein composition comprises performing a nuclear magnetic resonance (NMR) method.

46. The method of claim 38, wherein measuring the amount of antennary fucose present in the glycoprotein composition comprises performing monosaccharide analysis.

47. The method of claim 38, wherein measuring the amount of antennary fucose present in the glycoprotein composition comprises performing a fluorescence method.

48. The method of claim 38, wherein measuring the amount of antennary fucose present in the glycoprotein composition comprises performing a UV-VIS absorbance method.

49. The method of claim 38, wherein measuring the amount of antennary fucose present in the glycoprotein composition comprises performing an enzymatic method.

50. The method of claim 38, wherein measuring the amount of antennary fucose present in the glycoprotein composition comprises use of a detection molecule.

51. A method of producing a glycoprotein having a target level of antennary fucosylation, the method comprising (a) defining a target level of antennary fucosylation to be present in a therapeutic glycoprotein, (b) selecting a CHO cell as a host cell for production of the therapeutic glycoprotein if the target level of antennary fucosylation is greater than zero, (c) genetically engineering the selected CHO cell to express the therapeutic glycoprotein, and (d) culturing the genetically engineered CHO cell to produce the therapeutic glycoprotein, wherein the CHO cell is not genetically engineered or mutagenized to express an α3/α4 antennary fucosyltransferase.

52. The method of claim 51, further comprising, after step (a), screening CHO cells clones for a pre-specified level of antennary fucosylation.

53. The method of claim 51, further comprising, before step (a), measuring a level of antennary fucosyltransferase in a target glycoprotein or reference glycoprotein.

54. The method of claim 51, wherein the target level of antennary fucosylation is a level present in a commercial glycoprotein having at least 90% amino acid sequence identity to the therapeutic glycoprotein produced by the method.

55. The method of claim 51, wherein the target level of antennary fucosylation is a level greater than that present in a commercial glycoprotein having at least 90% amino acid sequence identity to the therapeutic glycoprotein produced by the method.

56. The method of claim 51, wherein the target level of antennary fucosylation is a level lower than that present in a commercial glycoprotein having at least 90% amino acid sequence identity to the therapeutic glycoprotein produced by the method.

57. The method of claim 1, further comprising measuring the level of antennary fucosylation in the produced glycoprotein.

58. A recombinant glycoprotein produced in CHO-K1 cells or a derivative thereof, wherein the recombinant glycoprotein has a different level of antennary fucosylation than a reference glycoprotein having at least 90% amino acid sequence identity.

59. The method of claim 58, wherein the reference glycoprotein is a commercially available therapeutic glycoprotein of Table 2.

60. An isolated population of CHO-K1 cells, wherein the CHO-K1 cells have not been genetically engineered or mutagenized to express an α3/α4 antennary fucosyltransferase, and wherein the population has been selected for high level expression of an α3/α4 antennary fucosyltransferase.
